(19) United States
(12) Patent Application Publication
(10) Pub. No.: US 2021/0236862 A1
(43) Pub. Date: Aug. 5, 2021

(54) TISSUE TREATMENT WITH SENSITIZER AND LIGHT AND/OR SOUND

(71) Applicant: CranioVation, Inc., Oakdale, MN (US)

(72) Inventor: Braden ELIASON, Minneapolis, MN (US)

(73) Assignee: CranioVation, Inc., Minneapolis, MN (US)

(21) Appl. No.: 17/266,157

(22) PCT Filed: Aug. 8, 2019
(86) PCT No.: PCT/US2019/045802

§ 371 Date: Feb. 5, 2021

Related U.S. Application Data
(60) Provisional application No. 62/716,153, filed on Aug. 8, 2018.

(51) Int. Cl. A61N 7/00; A61N 5/06

(52) U.S. Cl. CPC A61N 7/00; A61N 5/062; A61B 2090/033; A61N 2007/006; A61N 2007/0078; A61N 2007/0043

(57) ABSTRACT

A catheter is disclosed that may be used in a minimally invasive internal treatment (e.g., sonodynamic therapy). The catheter can include a housing, a portion of which may be positioned in contact with internal tissue of a patient during a minimally invasive sonodynamic or photo-sonodynamic therapy procedure. The catheter may include multiple electrically independent ultrasound transducers. The ultrasound transducers can be configured to emit ultrasound energy into the internal tissue of the patient. The ultrasound energy that is emitted from the catheter may reach a target tissue depth at a relatively low temporal average intensity (e.g., less than 50 W/cm2). Such ultrasound energy may activate the sensitizer.

### RELATED APPLICATIONS

The present application claims priority to U.S. Provisional Patent Application No. 62/716,153, filed Aug. 8, 2018, the entire contents of which is incorporated herein by reference.

### BACKGROUND

This disclosure relates to treating internal tissue by administering one or more sensitizers and exposing the tissue to light and/or ultrasound energy in a minimally invasive manner. The sensitizer is selected to preferentially accumulate inside unwanted cells in the tissue (e.g., cancer cells), and the light and/or sound activates the sensitizer, causing the sensitizer to kill the undesirable cells. Some of the minimally invasive procedures discussed herein involve photodynamic therapy, which uses only light to activate the sensitizer. Some of the minimally invasive procedures discussed herein involve sonodynamic therapy, which uses only ultrasound to activate the sensitizer. Some of the minimally invasive procedures discussed herein involve photo-sonodynamic therapy, which uses both light and ultrasound to activate the sensitizer.

### SUMMARY

This disclosure describes and illustrates catheters that can be used in complex, minimally invasive sonodynamic or photo-sonodynamic therapy procedures. Using sonodynamic or photo-sonodynamic therapy to kill brain tumors can be particularly challenging given accessibility obstacles and the desire to do no harm to healthy brain tissue. Catheters and techniques described herein can enable the use of sonodynamic and photo-sonodynamic therapy to achieve positive outcomes even for internal tissue that is hard to access.

In many embodiments, multiple ultrasound transducers are housed in a catheter housing and delivered to target internal tissue in a minimally invasive manner. The ultrasound transducers can emit ultrasound energy, which penetrates deeper into tissue than light, to activate one or more sensitizers to kill undesirable cells. Each ultrasound transducer may be separately connected to a power supply and can be independently electrically stimulated. The ultrasound energy emission pattern emitted by the catheter can be controlled by stimulating different individual ultrasound transducers differently. For example, the amplitude or phase of electrical stimulation may vary from one ultrasound transducer to another. Shaping and adjusting the ultrasound energy emission pattern can provide significant advantages in delivering ultrasound energy to internal tissue that is difficult to access. As alluded to, this can be especially beneficial in killing brain tumors minimally invasively.

In some embodiments, a catheter may be used in a minimally invasive internal treatment (e.g., sonodynamic therapy). The catheter can include a housing that has proximal and distal ends. A portion of the housing may be configured to be positioned in contact with internal tissue of a patient during a minimally invasive procedure that involves a sensitizer. The catheter may include multiple conductive pairs that are housed by the housing and that extend between the proximal and distal ends. Each conductive pair may have a first end that is configured to be connected to a power supply and a second end. The catheter may also include multiple ultrasound transducers housed by the housing. Each ultrasound transducer may be connected to the second end of a corresponding conductive pair. The ultrasound transducers can be configured to emit ultrasound energy independently into the internal tissue of the patient. The ultrasound energy that is emitted from the catheter may reach a target tissue depth at a relatively low temporal average intensity (e.g., less than 50 W/cm²). Such ultrasound energy may activate the sensitizer during the minimally invasive procedure.

### BRIEF DESCRIPTION OF DRAWINGS

The following drawings are illustrative of particular embodiments of the present invention and therefore do not limit the scope of the invention. The drawings are not necessarily to scale (unless so stated) and are intended for use in conjunction with the explanations in the following description. Embodiments of the invention will hereinafter be described in conjunction with the appended drawings, wherein like numerals denote like elements.

FIG. 1A is two-dimensional orthographic view of an illustrative catheter for performing sonodynamic therapy with an ultrasound transducer and a treatment field.

FIG. 1B is a cutaway, top elevational view of an illustrative catheter with an ultrasound transducer having a first emitting surface oriented non-parallelly with the catheter axis at an angle, Θ.

FIG. 2A is a cutaway, side elevational view of an illustrative catheter with a tube ultrasound transducer and a treatment field.

FIG. 2B is a cutaway, top elevational view of the catheter of FIG. 2A.

FIG. 3A is a cutaway, side elevational view of an illustrative catheter with a spherical shell ultrasound transducer.

FIG. 3B is a side elevational, cross-sectional view of the ultrasound transducer of FIG. 3A.

FIG. 3C is a perspective view of the ultrasound transducer of FIG. 3A.

FIG. 4A is a cutaway, side elevational view of an illustrative catheter with a flat ultrasound transducer and a treatment field.

FIG. 4B is a cutaway, front elevational view of the catheter of FIG. 4A.

FIG. 5A is a cutaway, side elevational view of an illustrative catheter with a flat ultrasound transducer being pivoted and a treatment field.

FIG. 5B is a cutaway, top elevational view of an illustrative catheter with a flat ultrasound transducer being pivoted and a treatment field.

FIG. 6A is a perspective view of an illustrative flat ultrasound transducer including first and second passive shells.

FIG. 6B is a cutaway, side elevational view of an illustrative catheter with the ultrasound transducer of FIG. 6A.

FIG. 6C is an exploded, side elevational view of the ultrasound transducer of FIG. 6A.

FIG. 7A is a perspective view of an illustrative ultrasound transducer that is a stack with first and second flat transducers and first and second passive shells.

FIG. 7B is a cutaway, side elevational view of an illustrative catheter with the ultrasound transducer of FIG. 7A.

FIG. 7C is an exploded side elevational view of the ultrasound transducer of FIG. 7A.

FIG. 8 is a cutaway, side elevational view of an illustrative catheter with multiple ultrasound transducers including first, second, and third flat transducers and a treatment field.

FIG. 9A is a perspective view of an illustrative ultrasound transducer including first, second, and third flat transducers and first and second passive shells.

FIG. 9B is a cutaway, side elevational view of an illustrative catheter with the ultrasound transducer of FIG. 9A.

FIG. 9C is an exploded, side elevational view of the ultrasound transducer of FIG. 9A.

FIG. 10A is a cutaway perspective view of an illustrative catheter with an array of ultrasound transducers.

FIG. 10B is a cutaway, side elevational view of the catheter of FIG. 10B.

FIG. 10C is a cutaway, side elevational view of an illustrative catheter with an array of ultrasound transducers connected to a power supply through a conductive pair.

FIG. 10D is a cutaway, side elevational view of an illustrative catheter with an array of ultrasound transducers that are each connected to a power supply by their own individual conductive pair.

FIG. 11 is a cutaway, side elevational view of an illustrative catheter with an ultrasound transducer and an acoustic element and a treatment field.

FIG. 12A is a partial side elevational view of an illustrative catheter for performing photodynamic therapy with an optical fiber connected to a light supply and an optical element.

FIG. 12B is a partial side elevational view of an optical fiber and an optical element for an illustrative catheter.

FIG. 12C is a partial side elevational view of an optical fiber and an optical element that is a shaped tip for an illustrative catheter.

FIG. 13A is a partial side elevational view of an illustrative catheter for performing sono-photodynamic therapy with an ultrasound transducer, an optical fiber connected to a light supply, and an optical element.

FIG. 13B is a cutaway, side elevational view of an illustrative catheter with an ultrasound transducer and an optical element positioned proximal to the ultrasound transducer.

FIG. 13C is a cutaway, side elevational view of an illustrative catheter with an ultrasound transducer and an optical element positioned distal to the ultrasound transducer.

FIG. 13D is a cutaway, side elevational view of an illustrative catheter with an ultrasound transducer, an acoustic element, and an optical element positioned distal to the ultrasound transducer and the acoustic element.

FIG. 13E is a cutaway, side elevational view of an illustrative catheter with an ultrasound transducer, an acoustic element, and an optical element positioned distal to the ultrasound transducer and proximal to the acoustic element.

FIG. 13F is a cutaway, side elevational view of an illustrative catheter with an ultrasound transducer, an acoustic element, and an optical element positioned proximal to the ultrasound transducer and the acoustic element.

FIG. 14A is a flow chart of an illustrative method for emitting either or both of ultrasound energy and light into the internal tissue of a patient.

FIG. 14B is a flow chart continuing from the flow chart in FIG. 14A of an illustrative method for emitting either or both of ultrasound energy and light into the internal tissue of a patient.

FIG. 15 is a cutaway perspective view of an illustrative catheter with a tube ultrasound transducer being rotated.

FIG. 16 is a cutaway, front elevational view of an illustrative catheter with a tube ultrasound transducer being rotated and a treatment field.

FIG. 17 is a cutaway, side elevational view of an illustrative catheter being rotated with a flat ultrasound transducer being pivoted and a treatment field.

FIG. 18 is a cutaway, side elevational view of an illustrative catheter with multiple ultrasound transducers including a first, second, and third flat transducer and a treatment field using a beamforming technique.

FIG. 19A shows an embodiment of an acoustic lens.

FIG. 19B shows an embodiment of an acoustic lens.

FIG. 19C shows an embodiment of an acoustic lens.

FIG. 19D shows an embodiment of an acoustic lens.

FIG. 20A is a schematic view of an illustrative catheter.

FIG. 20B is a schematic view of an illustrative catheter.

FIG. 20C is a schematic view of an illustrative catheter.

FIG. 21A shows a two-dimensional array of ultrasound transducers.

FIG. 21B shows a two-dimensional array of ultrasound transducers.

FIG. 21C shows a two-dimensional array of ultrasound transducers.

FIG. 21D shows a two-dimensional array of ultrasound transducers.

FIG. 22 is a schematic view of an illustrative catheter.

FIG. 23A is two-dimensional orthographic view of a catheter used in connection with a stereotactic guidance system.

FIG. 23B is two-dimensional orthographic view of a catheter used in connection with a stereotactic guidance system.

### DETAILED DESCRIPTION

The following detailed description is exemplary in nature and is not intended to limit the scope, applicability, or configuration of the invention in any way. Rather, the following description provides some practical illustrations for implementing exemplary embodiments of the present invention. Examples of constructions, materials, and/or dimensions are provided for selected elements. Those skilled in the art will recognize that many of the noted examples have a variety of suitable alternatives.

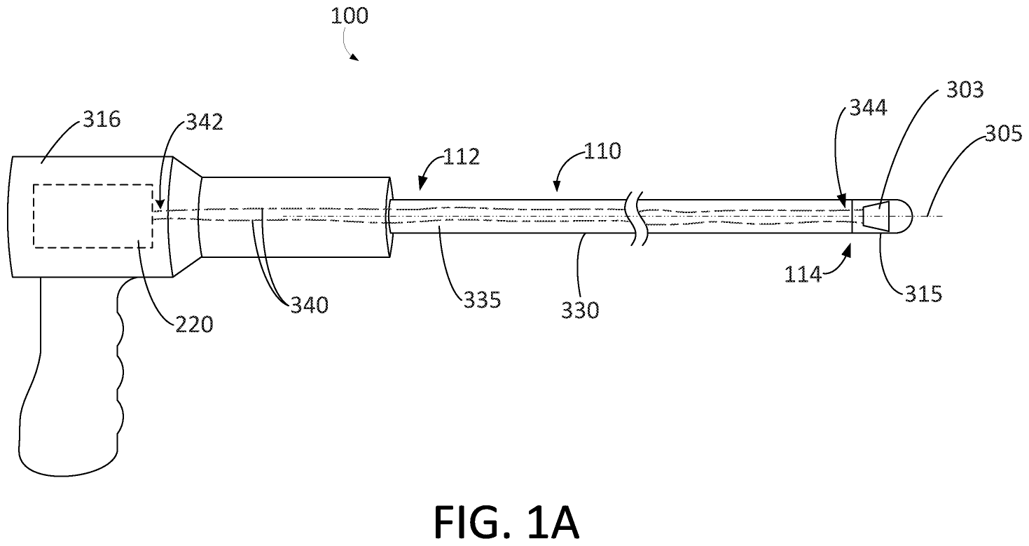

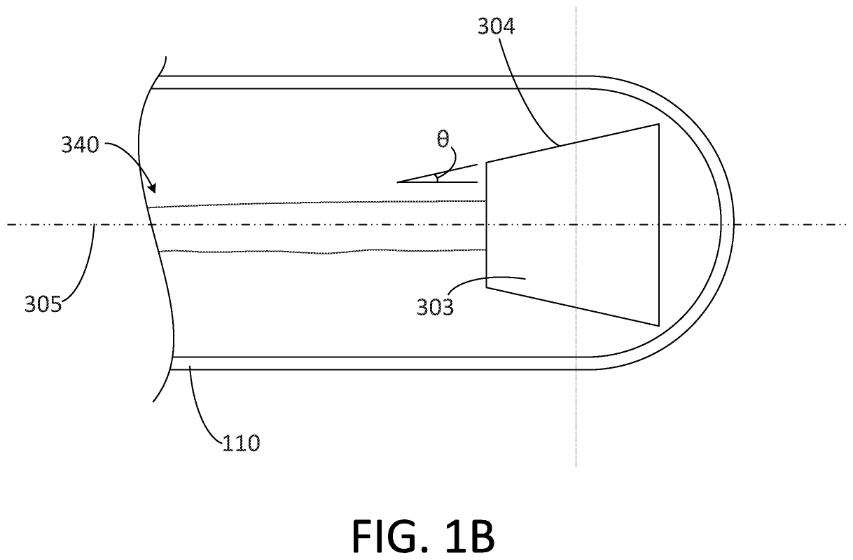

FIG. 1A shows an illustrative catheter 100 that can be used for minimally invasive sonodynamic therapy. The housing 110 can include a proximal end 112 and a distal end 114. The housing 110 can define a catheter axis 305. In various instances, the housing 110 can be a flexible elongate member. The housing 110 in some embodiments can be formed of a flexible material such as plastic. In many embodiments, the housing 110 can be a catheter. In some embodiments, the housing 110 can have a solid cross section with components of the catheter 100 integrally manufactured (e.g., injection molded) together. In some embodiments, the housing 110 can be of relatively rigid construction. In certain instances, the housing 110 can have a tubular cross section as described elsewhere herein. The catheter axis 305 can extend the length of the housing 110. In some embodiments, the catheter axis 305 can be located along the longitudinal centerline of the housing 110.

At least a portion of the housing 110 can be configured to be in contact with portions of the patient's body. A portion of the housing 110 can be configured to be positioned in contact with internal tissue of a patient. In some embodiments, the portion of the housing 110 that contacts the internal tissue can be configured to be positioned intracranially in contact with brain tissue of the patient. In many embodiments, the portion of the housing 110 can include the distal end 114 of the housing 110 (e.g., the tip of the distal end 114). The portion of the housing 110 that contacts the internal tissue may include a transducer housing 315 as described elsewhere herein.

The housing 110 of the illustrative catheter 100 can be adapted to be used during minimally invasive procedures. In some embodiments, the portion of the housing 110 that can be configured to be positioned in contact with internal tissue of a patient can have a cross-sectional area of less than 154 mm² (e.g., a tube with a diameter of 14 mm or less). In many instances, as described elsewhere herein, the minimally invasive procedure can include sonodynamic therapy. In some instances, the minimally invasive procedure can include photodynamic therapy. In certain circumstances, the minimally invasive procedure can include photo-sonodynamic therapy.

In many embodiments, the catheter 100 can be used to perform a minimally invasive procedure on the brain of a patient. In various instances, minimally invasive photodynamic and/or sonodynamic therapy can be performed on the brain tissue of a patient. In many embodiments, a portion of the housing 110 can be configured to be positioned intracranially in contact with brain tissue of the patient during the minimally invasive procedure.

In some embodiments, the housing 110 can include a sheath 330. In many embodiments, the sheath 330 can extend the length of the housing 110. The sheath 330 in various embodiments can include a wall at the periphery of the housing 110. In some embodiments, the wall can be a thin wall.

The sheath 330 can define a lumen 335. The lumen 335 can extend along the catheter axis 305. The lumen 335 in various embodiments can extend along the length of the housing 110. The lumen 335 can extend along the longitudinally centerline of the housing 110 in various instances. In many embodiments, the lumen 335 can have a cross-sectional area that is large enough to house an optical element and/or an ultrasound transducer.

At least a portion of the housing 110 in many embodiments can be used to house the ultrasound transducer 303. The housing 110 can include a transducer housing 315. The transducer housing 315 can house the ultrasound transducer 303. The transducer housing 315 can be integral to other portions of the housing 110 in some instances. In some instances, the transducer housing 315 can be separately connected to the housing 110. In many embodiments, the transducer housing 315 can be more rigid than other portions of the housing 110. The geometric profile of the transducer housing 315 may vary from that of the other portions of the housing 110 in some instances and may be the same in other instances. The transducer housing 315 can have an acoustic impedance that is similar to the acoustic impedance of the surrounding tissue. In some instances, the transducer housing 315 can have an acoustic impedance that is between the acoustic impedance of the surrounding tissue and the acoustic impedance of the ultrasound transducer 303. In many embodiments, the transducer housing 315 can have a relatively high electromagnetic impedance to serve as an insulator. For example, the transducer housing 315 can include a potting material with a high volume resistivity (e.g., between 10⁸ Ohms-cm and 10¹⁸ Ohms-cm, such as 10¹² Ohms-cm or 10¹⁴ Ohms-cm).

In some embodiments, an acoustic transmission material can be positioned between the ultrasound transducer(s) and where ultrasound energy exits the housing into the internal tissue of the patient. In catheters that have a transducer housing for housing the ultrasound transducer(s), acoustic transmission material may include a fluid couplant that fills the transducer housing. In some embodiments, acoustic transmission material may include one or more acoustic matching layers coated on one or more ultrasound transducers. In some embodiments, acoustic transmission material may include an elastic boot.

The catheter 100 can include a handle 316. In some embodiments, the housing 110 can include the handle 316. In some embodiments, the handle 316 can be separate from and connected to the housing 110. The handle 316 can be positioned at the proximal end 112 of the housing 110. The handle 316 can be adapted to fit within the hand of a user. In some embodiments, the handle 316 can include features (e.g., one or more grips or a patient interface module) which can facilitate moving components of the catheter 100.

The illustrative catheter 100 can include a conductive pair 340 that can be adapted to connect to other components of the catheter 100. The conductive pair 340 can be housed by the housing 110. The conductive pair 340 can have a first end 342 and a second end 344. The conductive pair 340 can extend between the proximal end 112 and the distal end 114 of the housing 110. The conductive pair 340 in some embodiments can be wires made from conductive materials (e.g., aluminum or copper). In some embodiments, the conductive pair 340 can extend within the lumen 335 of the sheath 330. In some embodiments, the conductive pair 340 can be positioned along the centerline of the housing 110. In some embodiments, the conductive pair 340 can be positioned away from the centerline of the housing 110 (e.g., offset or about the periphery of the lumen). As described elsewhere herein, in some instances, the conductive pair 340 can include a stem portion and one or more branch portions.

The first end 342 of the conductive pair 340 can be configured to be connected to a power supply 220. In some embodiments, the power supply 220 can be a wire configured to connect to an external power supply (e.g., an external treatment console). In some embodiments, for example, when the power supply 220 is internal to the handle 316, the conductive pair 340 can be connected to the power supply 220 via connective joints (e.g., soldering, serial bus, or the like).

The ultrasound transducer 303 can be connected to the second end 344 of the conductive pair 340. The ultrasound transducer 303 can be housed by the housing 110. The ultrasound transducer 303 can take a variety of forms and shapes as described elsewhere herein. In some embodiments, the ultrasound transducer 303 can be housed in the transducer housing 315 as described elsewhere herein. In many embodiments, the conductive pair 340 can be connected to the ultrasound transducer 303 in a similar manner as the conductive pair 340 is connected to the power supply 220. The ultrasound transducer 303 can be made of a suitable material, e.g., having piezo-electric properties.

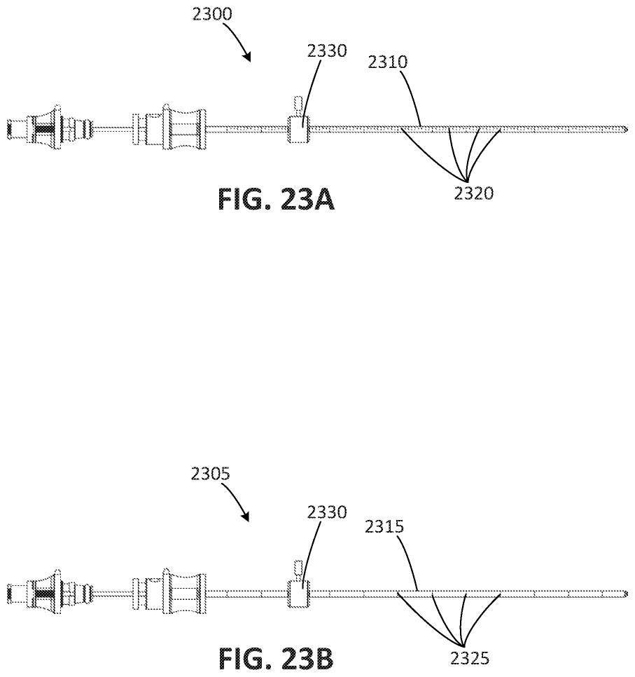

FIGS. 23A-23B show catheters 2300, 2305 that can be used in connection with a stereotactic guidance system. The catheters 2300, 2305 can have characteristics and functionality of other catheters discussed herein. The catheters 2300, 2305 may each include a housing 2310, 2315. The housings 2310, 2315 can each have one or more location markers 2320, 2325 for use with a stereotactic guidance system. In some embodiments, the location markers 2320, 2325 may facilitate measuring a distance from a reference point in a stereotactic guidance system. As can be seen, the location markers 2320 of FIG. 23A are more granular than the location markers 2325 of FIG. 23B. In some embodiments, the catheters 2300, 2305 may include an adjustable depth stop 2330 configured to slide over the housing 2310, 2315 and to be locked in different locations for use with a stereotactic guidance system. Using a stereotactic guidance system can enable precise positioning of a catheter 2300, 2305 within internal tissue for minimally invasive sonodynamic or photo-sonodynamic therapy.

The ultrasound transducer 303 in the illustrative catheter 100 can include a first emitting surface 304 as shown in FIG. 1B. The first emitting surface 304 can be oriented non-parallelly with the catheter axis 305. The first emitting surface 304 can be positioned at an angular distance Θ from the catheter axis 305. Although the first emitting surface 304 is shown at a specific angle, Θ can be any angle such that the first emitting surface 304 is positioned non-parallelly with the catheter axis 305 (e.g., 5°, 15°, 30°, 45°, 90°, etc.). Accordingly, in some embodiments, the first emitting surface 304 is positioned to be perpendicular to the catheter axis 305. In some embodiments, the first emitting surface 304 can be at an acute or obtuse angle relative to the catheter axis 305. The first emitting surface 304 can be any surface on the ultrasound transducer 303 that is positioned non-parallelly with the catheter axis 305 and that emits ultrasound energy. In many embodiments, the first emitting surface 304 can be an outer surface of the ultrasound transducer 303. The first emitting surface 304 may be a variety of shapes and forms as discussed elsewhere herein.

In many embodiments, the first emitting surface 304 of the ultrasound transducer 303 can be configured to emit ultrasound energy. The ultrasound energy can be emitted into the internal tissue of the patient. The ultrasound energy can be emitted into the internal tissue of the patient during the minimally invasive procedure. In some embodiments, the ultrasound energy can be emitted into the brain tissue of the patient.

As shown in FIG. 1B (and throughout several of the figures) indicated by the dashed arrows, the ultrasound energy can be emitted such that it radiates outwardly from the ultrasound transducer 303. The ultrasound energy can be emitted beyond the housing 110 so as to generate a treatment field. The treatment field can have a variety of shapes and forms as shown throughout the figures and as described elsewhere herein. In many instances, for example, any of the size, shape, or form of the treatment field can correspond to the type of ultrasound transducer 303 emitting the ultrasound energy.

In many embodiments, the ultrasound energy emitted by the first emitting surface 304 can be at a low intensity. Intensity can be measured in various ways. For example, intensity may be measured as an average intensity over time—a temporal average intensity. Other ways to measure intensity include as a pulse averaged intensity, spatial-peak intensity, and spatial-average intensity. In many instances, ultrasound energy can be emitted such that it reaches a target tissue depth at a temporal average intensity of less than 50 W/cm². In some embodiments, ultrasound energy can be emitted by the first emitting surface 304 such that it reaches a target tissue depth at even lower temporal average intensities, such as less than 25 W/cm², less than 10 W/cm², less than 5 W/cm², or less than 3 W/cm². For example, the temporal average intensity may be 60 W/cm² or 120 W/cm² immediately next to the ultrasound transducer 303, with the temporal average intensity decreasing as the distance from the ultrasound transducer 303 increases. When the distance from the ultrasound transducer 303 reaches the target tissue depth, the temporal average intensity can be below an ablation threshold. In this way, such non-ablative ultrasound energy (low intensity for short duration) can minimize or eliminate the impact on tissue not targeted for treatment while killing the undesirable cells by activating the sensitizer.

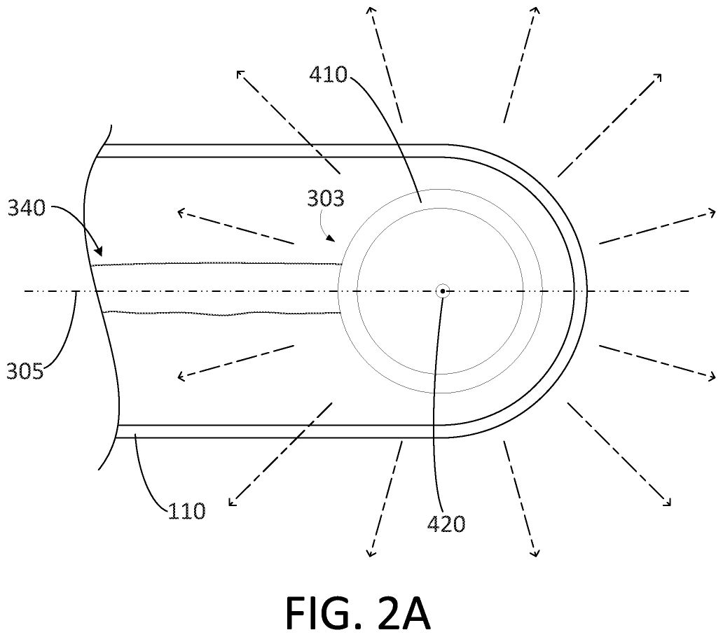

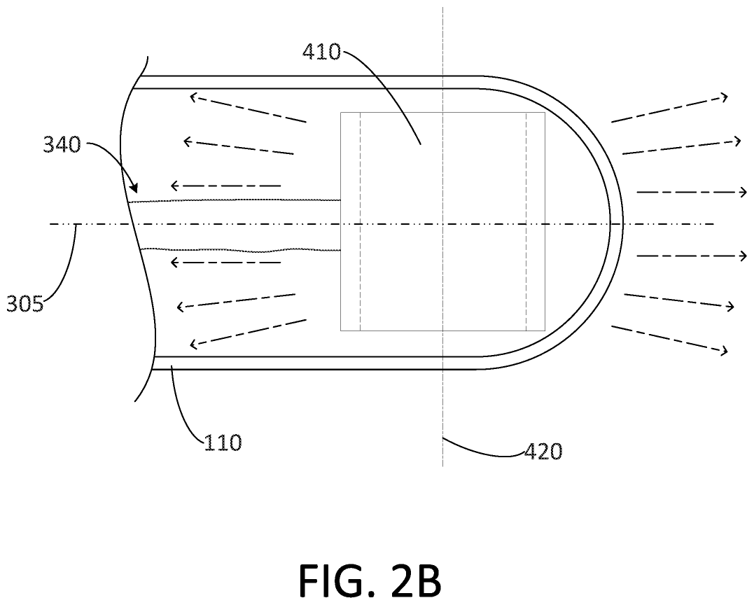

In some embodiments, the ultrasound transducer 303 can include a tube 410 as shown in FIGS. 2A and 2B. The tube 410 can have a tube axis 420. In some embodiments, the ultrasound transducer 303 can be oriented such that the tube axis 420 is noncoaxial to the catheter axis 305. In some embodiments, the ultrasound transducer 303 can be oriented such that the tube axis 420 is perpendicular to the catheter axis 305. In many embodiments, the tube axis 420 can extend along the longitudinal centerline of the tube. As shown in FIG. 2A, the ultrasound energy emitted from the tube 410 can radiate outwardly beyond the outer surface of the tube to form an elongated treatment field.

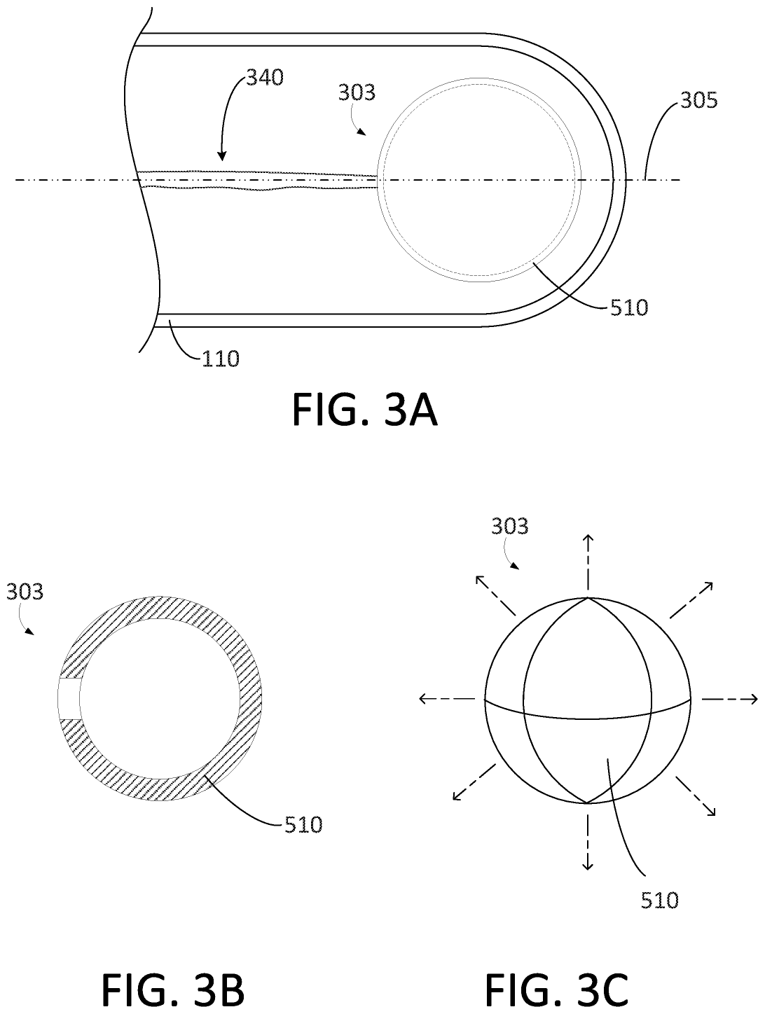

In some embodiments, the first emitting surface can include a spherical shell 510 as shown in FIGS. 3A-3C. As shown in FIG. 3C, the ultrasound energy emitted by the spherical shell 510 can radiate outwardly from the first emitting surface of the spherical shell 510 to form a spherical treatment field. The spherical shell 510 can have an outer wall. The outer wall can surround an internal cavity of the spherical shell 510.

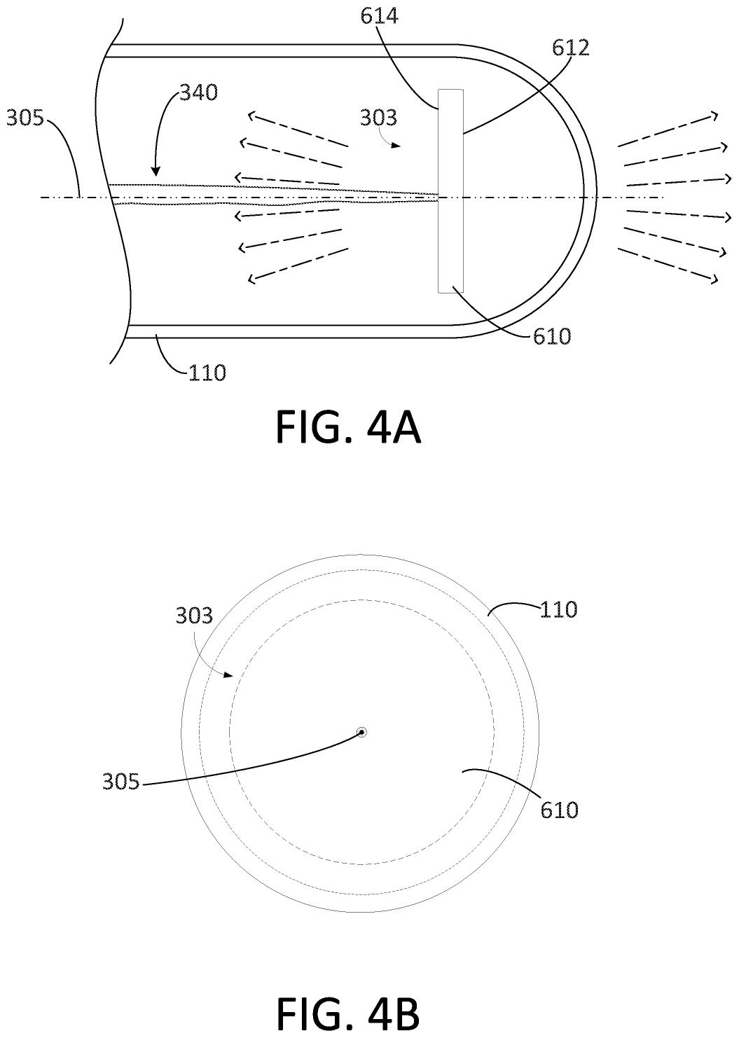

In some embodiments, the ultrasound transducer 303 can include a flat transducer 610 as shown in FIGS. 4A and 4B. As shown in FIG. 4A, ultrasound energy can radiate outwardly from the first emitting surface 612 of the flat transducer 610 beyond the housing to form a conical or frustum-shaped treatment field. In some embodiments, the ultrasound transducer 303 can include a disk as shown in FIG. 4B.

The flat transducer 610 can include emitting surfaces as shown in FIGS. 4A and 4B. The flat transducer 610 can include a first emitting surface 612. The flat transducer 610 can include a second emitting surface 614. The second emitting surface 614 can be opposite the first emitting surface 612. The second emitting surface 614 can operate similarly as described for the first emitting surface 612.

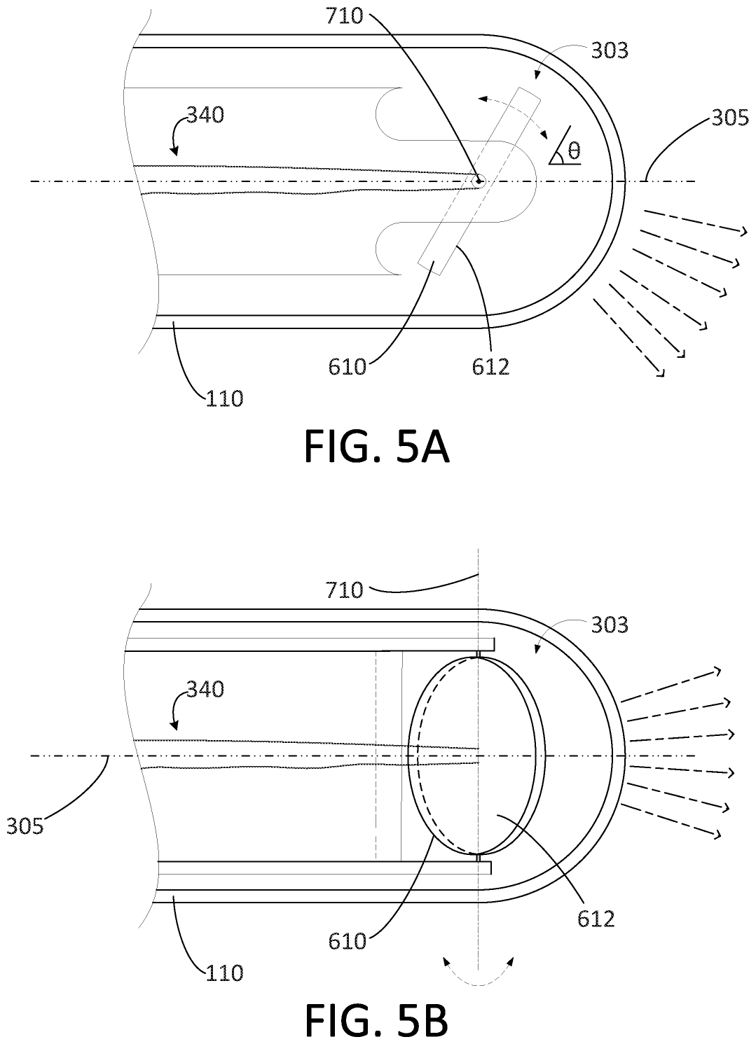

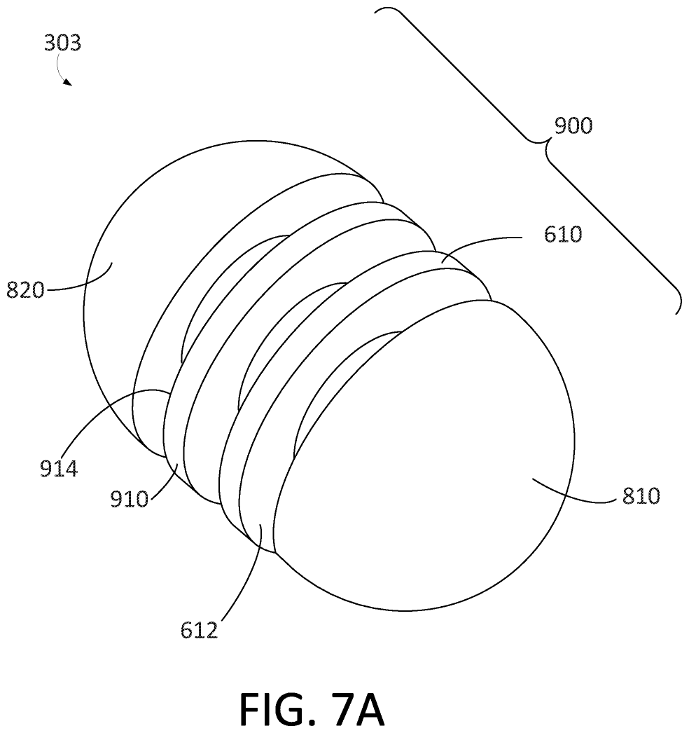

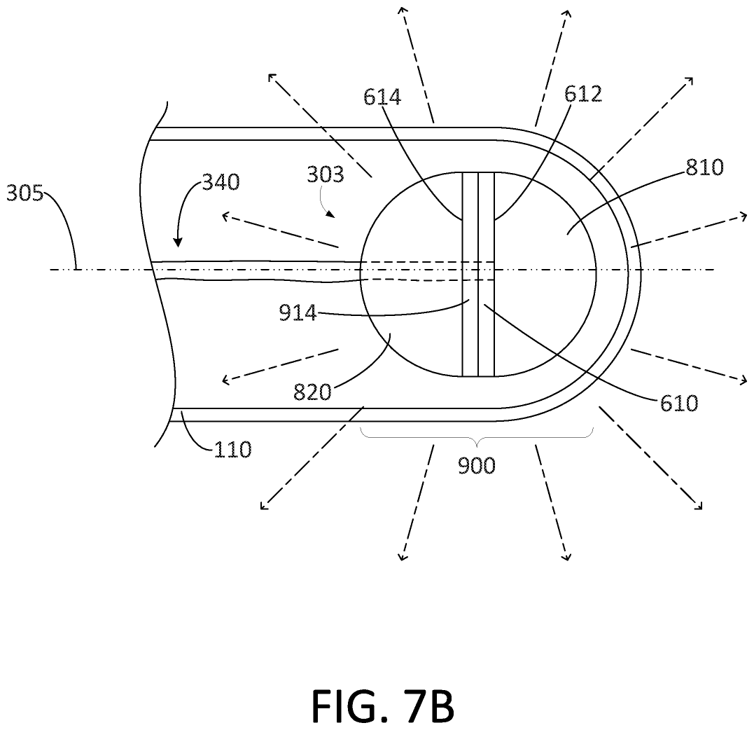

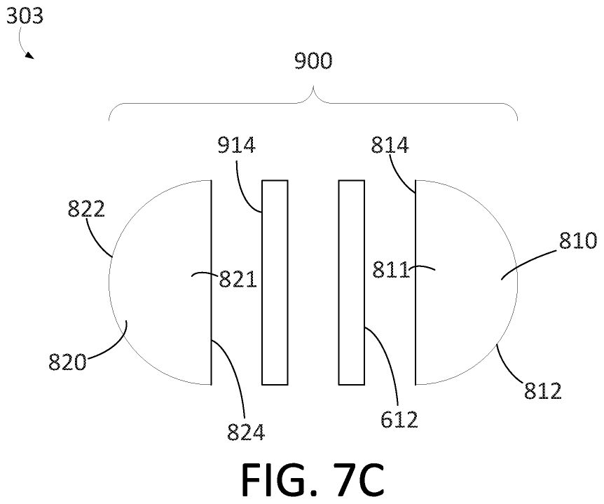

In some embodiments, the flat transducer 610 can be pivotable as shown in FIGS. 5A and 7B. The flat transducer 610 can be pivotable about a pivot axis 710 as indicated by the dashed double arrow in the figures. In many embodiments, the pivot axis 710 can extend along the centerline of the flat transducer 610. The flat transducer 610 can be pivotable relative to the housing 110 as shown in FIG. 5A. In some embodiments, the pivot axis 710 can be perpendicular to the catheter axis 305 as shown in FIG. 5B.

The flat transducer 610 in many embodiments can be pivoted about the pivot axis 710 while emitting ultrasound energy. Such a configuration, for example, can pivot the treatment field of the flat transducer 610 about the pivot axis 710. Accordingly, the treatment field can have an elongated profile about the pivot axis 710. In many embodiments, the flat transducer 610 can pivot 360 degrees around the pivot axis 710.

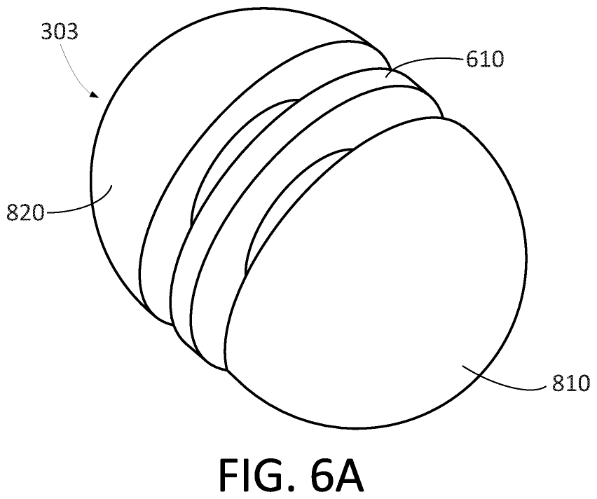

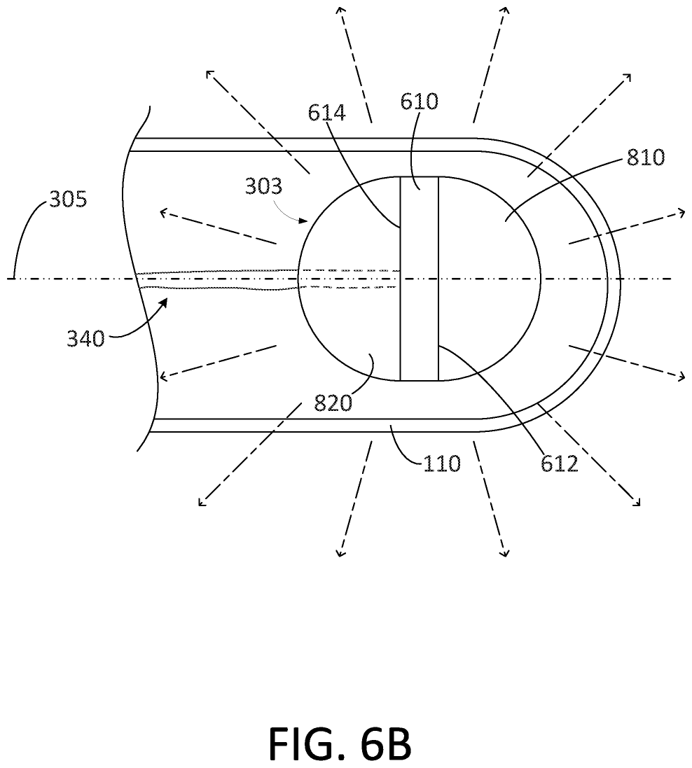

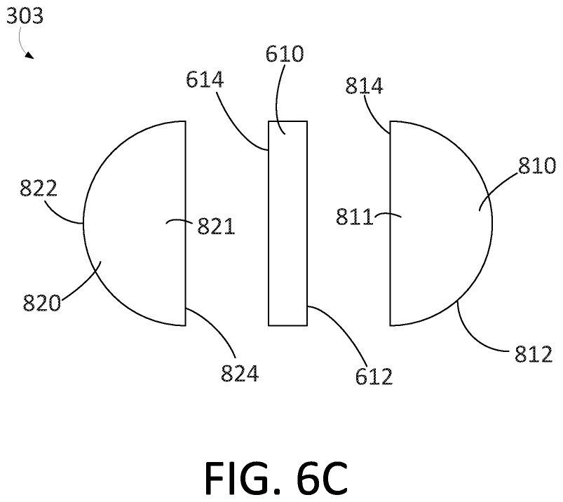

In some embodiments, the ultrasound transducer 303 can include a flat transducer 610, a first passive shell 810, and a second passive shell 820 as shown in FIGS. 6A-6C. As shown in FIG. 6A, the flat transducer 610 and first and second passive shells 810, 820 can be positioned such that they are coaxially aligned. In several embodiments, the flat transducer 610 and first and second passive shells 810, 820 can be mechanically connected, for example, using an adhesive, adjustable fasteners, or permanent fasteners.

Ultrasound energy from the ultrasound transducer 303 that includes a flat transducer 610, a first passive shell 810, and a second passive shell 820 can be emitted as shown in FIG. 6A. In many embodiments, the first and second passive shells 810, 820 are configured to transmit ultrasound energy emitted from the flat transducer 610. In this way, in various embodiments, the first and second passive shells 810, 820 may modify the treatment field of the flat transducer 610. In many instances, such a configuration can have a parabolic (e.g., spherical, ellipsoidal, etc.) treatment field on either side of the flat transducer 610. In any of these instances, the axial position of the first and second passive shells 810, 820 relative to the centerline of the flat transducer 610 can be altered to modify the treatment field.

The first and second passive shells 810, 820 can be positioned to be in contact with the flat transducer 610 as shown in FIG. 6B. The first passive shell 810 can be positioned in contact with the first emitting surface 612. The second passive shell 820 can be positioned in contact with the second emitting surface 614.

As shown in FIG. 6C, in some embodiments, the first passive shell 810 can include a first hemisphere 811. The first hemisphere 811 can include a first curved surface 812. The first hemisphere 811 can include a first flat surface 814. The first flat surface 814 can be positioned in contact with the first emitting surface 612. The first flat surface 814 can be positioned with the first curved surface 812 extending away from the first emitting surface 612.

In some embodiments, the second passive shell 820 can include a second hemisphere 821 as also shown in FIG. 6C. The second hemisphere 821 can include a second curved surface 822. The second hemisphere 821 can include a second flat surface 824. The second flat surface 824 can be positioned in contact with the second emitting surface 614. The second flat surface 824 can be positioned with the second curved surface 822 extending away from the second emitting surface 614.

In some embodiments, the ultrasound transducer 303 can include a stack 900 as shown in FIGS. 7A-7C. The stack 900 can include a first flat transducer 610. The stack 900 can include a second flat transducer 910. In many instances, the first and second flat transducer 910 can be similar to the flat transducer 610 described elsewhere herein. In some embodiments, the first flat transducer 610 is similar to the second flat transducer 910. The second flat transducer 910 may be different from the first flat transducer 610 in several embodiments. The stack 900 can be positioned and connected in a manner similar to the embodiments that can have a flat transducer 610 and first and second passive shells 810, 820 described elsewhere herein.

Ultrasound energy can be emitted from the ultrasound transducer 303 that includes a first flat transducer 610, a second flat transducer 910, a first passive shell 810, and a second passive shell 820 as shown in FIG. 7A. In such instances, the emission of ultrasound can have a similar profile to that of embodiments that have a flat transducer 610 and first and second passive shells 810, 820 as described elsewhere herein. In some embodiments, the addition of the second flat transducer 910 can affect the ultrasound emission relative to the embodiments that have a flat transducer 610 and first and second passive shells 810, 820. For instance, the treatment field may be enlarged or can shrink, the intensity of the emission may be increased or decreased, or the like.

The first and second flat transducers 610, 910 of the stack 900 can be positioned relative to one another as shown in FIG. 7B. The first flat transducer 610 can include the first emitting surface 612. The second flat transducer 910 can include a second emitting surface 914. The first and second flat transducers 610, 910 can be positioned such that the first and second emitting surfaces 612, 914 are opposite one another.

In some embodiments, the stack 900 can include first and second passive shells 810, 820 with respective first and second hemispheres 811, 821 as shown in FIG. 7C. The first passive shell 810 can be positioned in contact with the first emitting surface 612. The second passive shell 820 can be positioned in contact with the second emitting surface 914. In some embodiments, the first passive shell 810 of the stack 900 can include a first hemisphere 811 similar to those described elsewhere herein. In some embodiments, the second passive shell 820 of the stack 900 can include a second hemisphere 821 similar to those described elsewhere herein.

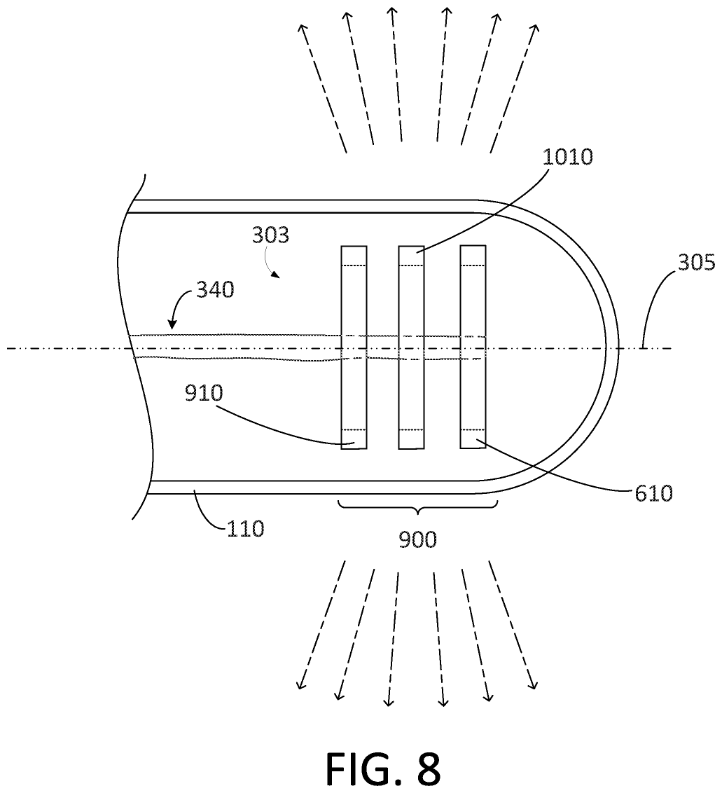

As shown in FIG. 8, in many instances, the stack 900 can include a third flat transducer 1010. In some embodiments, the third flat transducer 1010 can be positioned between the first and second flat transducers 610, 910. In many instances, the stack 900 can have any number of flat transducers 610. Such configurations may facilitate certain treatment field characteristics (e.g., beamforming) as described elsewhere herein. The location of the third flat transducer 1010, however, may vary between embodiments.

Ultrasound energy from the ultrasound transducer 303 that includes a first, second, and third flat transducer 610, 910, 1010 can be emitted as shown in FIG. 8. In such embodiments, the addition of a third flat transducer 1010 can modify the profile of the treatment field, the emission of ultrasound energy, or both similar to as described for the embodiments having first and second flat transducers 610, 910 and first and second passive shells 810, 820.

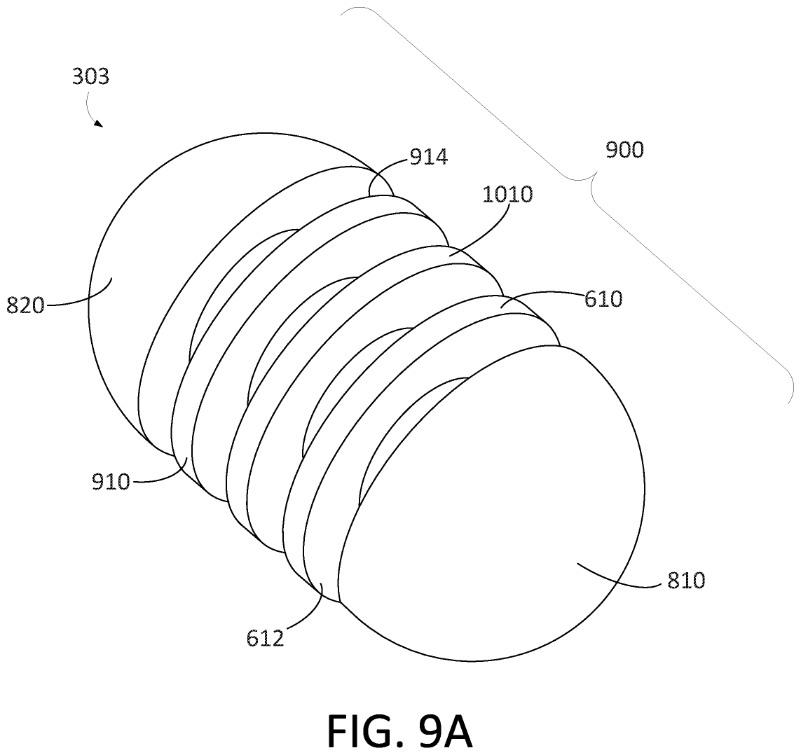

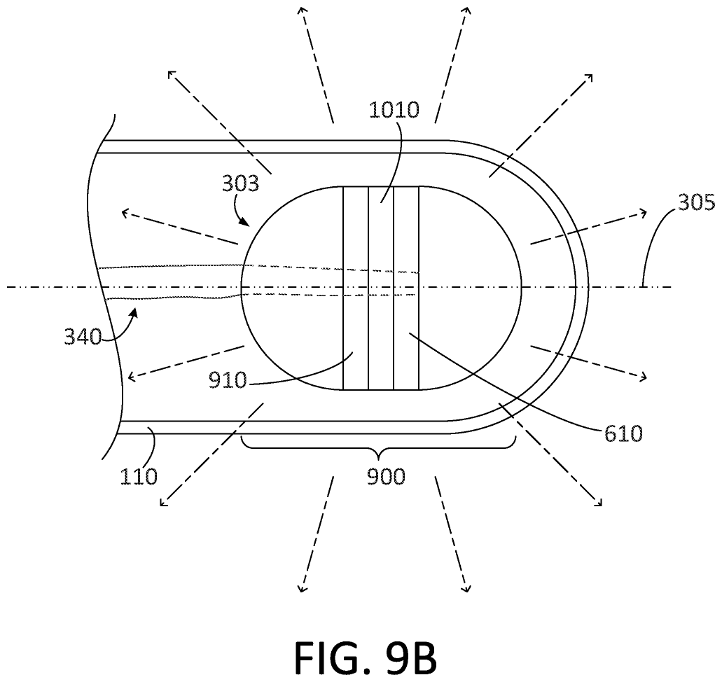

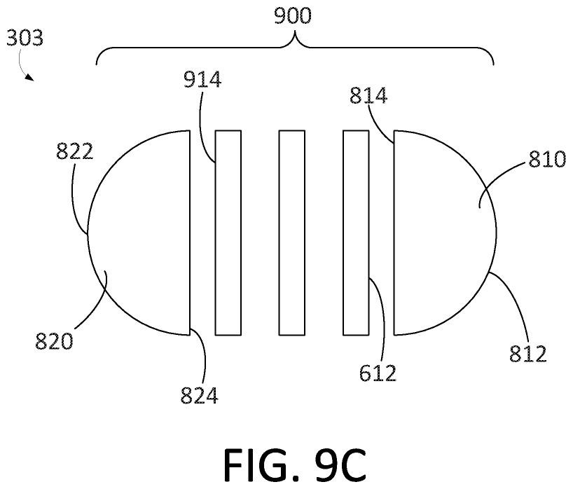

As shown in FIGS. 9A-9C, in many instances, the stack 900 can include a third flat transducer 1010 with first and second passive shells 810, 820. In many instances, the stack 900 can have any number of flat transducers 610 paired with first and second passive shells 810, 820. The flat transducers 610 and first and second passive shells 810, 820 can be positioned and operated as described elsewhere herein. Similarly, ultrasound emission can operate and be modified as described elsewhere herein.

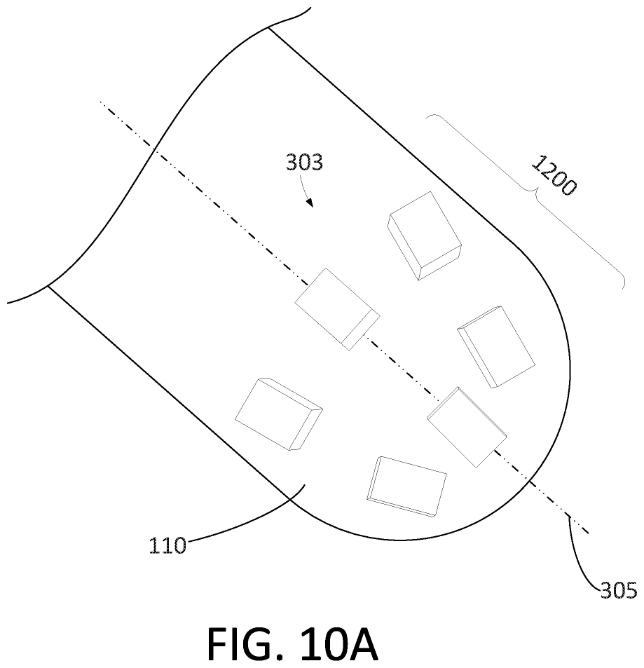

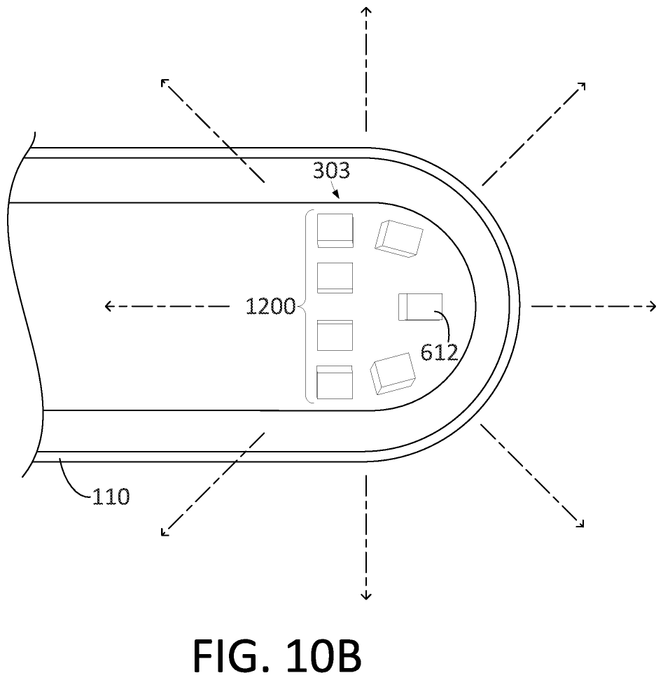

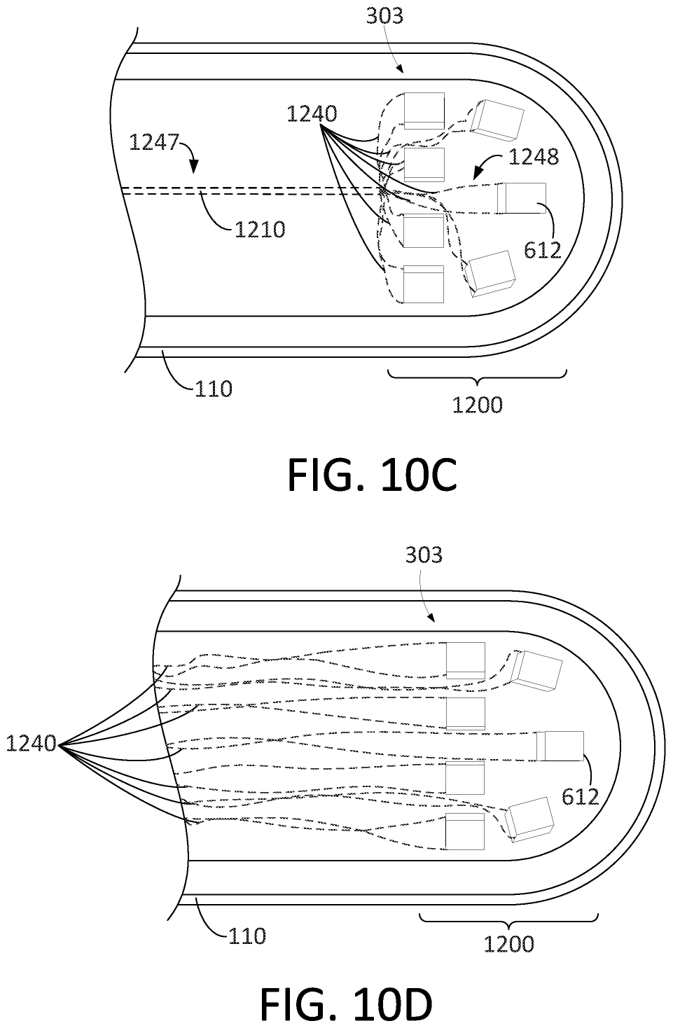

In some embodiments, the ultrasound transducer 303 can include an array 1200 of individual ultrasound transducers 303 as shown in FIGS. 10A-10D. The individual ultrasound transducers 303 in the array 1200 can be positioned about the distal end of the housing 110. In some embodiments, one or more of the individual ultrasound transducers 303 can be positioned at the tip at the distal end of the housing 110. The array 1200 may be mechanically attached to the housing 110, for example, using adhesives, removable or reusable fasteners, or permanent fasteners. In some embodiments, the array 1200 may be integral to the housing 110. Though depicted as rectangular, the shape of the individual ultrasound transducers 303 may vary between and within different embodiments.

One of the individual ultrasound transducers 303 in the array 1200 can include the first emitting surface 612 as shown in FIG. 10B. The first emitting surface 612 may be similar to those described elsewhere herein. Although the first emitting surface 612 is depicted on a particular individual ultrasound transducer 303 in the array 1200 and on a particular surface thereof, it should be noted that the first emitting surface 612 can be on any surface of any of the individual ultrasound transducers 303.

An illustrative catheter can convey power to the array 1200 of individual ultrasound transducers 303 as shown in FIGS. 10C and 10D. Each of the individual ultrasound transducers 303 can be electrically connected to the power supply. In some embodiments, each of the individual ultrasound transducers 303 can be electrically connected to the power supply through its own conductive pair 1240.

As shown in FIG. 10C, the conductive pair 1240 can include a stem portion 1247 and one or more branch portions 1248. The stem portion 1247 can comprise the first end of the conductive pair 1240. The one or more branch portions 1248 can comprise the second end of the conductive pair 1240. In some instances, the stem portion 1247 of the conductive pair 1240 can be composed of multiple conductive pairs 1240. In some instances, the stem portion 1247 of the conductive pair 1240 can be composed of a single conductive pair 1240 and a junction. In any of these embodiments, the one or more branch portions 1248 can be connected to each of the individual ultrasound transducers 303.

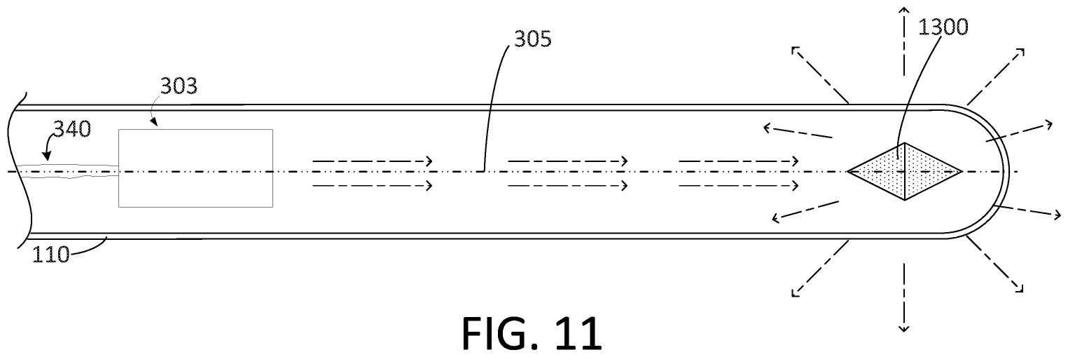

In many embodiments, the catheter can include an acoustic element 1300 as shown in FIG. 11. The acoustic element 1300 can be housed by the housing 110. In such embodiments, the acoustic element 1300 can be positioned distal to the ultrasound transducer 303. In many embodiments, the ultrasound transducer 303 can be positioned such that external to a patient during a procedure. In various embodiments, the acoustic element 1300 can be positioned such that it is internal to a patient during a procedure.

The acoustic element 1300 can be configured to modify a direction at which ultrasound energy emitted by the ultrasound transducer 303 enters the internal tissue of the patient. In such embodiments, the acoustic element 1300 can modify the direction of ultrasound energy emitted by the ultrasound transducer 303 during the minimally invasive procedure. As shown in FIG. 11, ultrasound energy can be emitted into the acoustic element 1300 by the ultrasound transducer 303. The acoustic element 1300 can receive the ultrasound energy and transmit (e.g., refracted, reflected, etc.) such that it radiates outwardly from the housing 110. The treatment field can vary depending on the geometry of the element in many instances, but may be similar to those described elsewhere herein. In some embodiments, the acoustic element 1300 can be housed in the transducer housing. Examples of acoustic elements include an acoustic lens, a waveguide, etc. The acoustic element can have the ability to redirect/reshape waves coming from the ultrasound transducer into a more desirable shape. For example, a planar wave from a flat transducer may be turned (e.g., 90 degrees) with an acoustic element. An acoustic lens may aid in focusing ultrasound waves onto a particular spot. In some instances, the acoustic lens may be in contact with (e.g., attached to) the emitting surface of the transducer to focus or defocus the acoustic wavefront formed by the ultrasound energy.

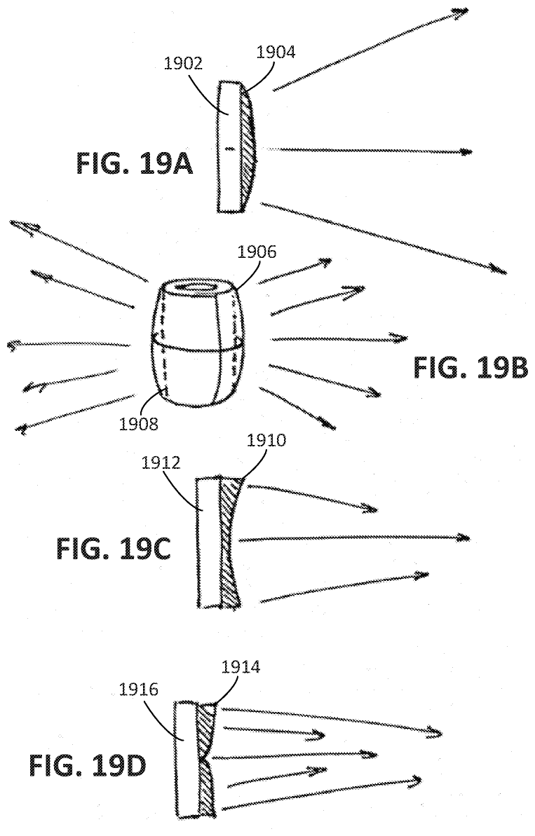

Examples of acoustic lenses are provided in FIG. 19A-19D. FIG. 19A shows a flat transducer 1902 with a lens 1904 that can produce a defocused (spread) wave front. FIG. 19B shows a lens 1906 on a cylinder 1908 that can produce a spread wave front. FIG. 19C shows a lens 1910 on a flat transducer 1912 that can produce a focused wave front. FIG. 19D shows a lens 1914 on a flat transducer 1916 that can produce a focused wave front.

In some embodiments, the acoustic element can be made of a material with a different speed of sound than its surroundings. To redirect the sound 90 degrees, two of the faces can be perpendicular, and the third wall can form the hypotenuse. The sound can be redirected because of total internal reflection. The sound can enter the first wall, reflect off of the hypotenuse, and exit the second wall. The sound can (mostly) reflect off the angled wall because the difference in the speed of sound between the element and its surrounding creates a critical angle. If the angle of incidence of the wave is greater than the critical angle, most of the wave is reflected, thereby changing its direction. Assuming materials of similar acoustic impedance, because the wave enters and exits the two perpendicular faces at an angle of incidence close to 0 degrees (less than the critical angle), the sound can enter the element instead of being reflected. To have a critical angle below 45°, the speed of sound of the element would need to be more than about 30% slower than its surroundings (a speed ratio of less than 1/√2).

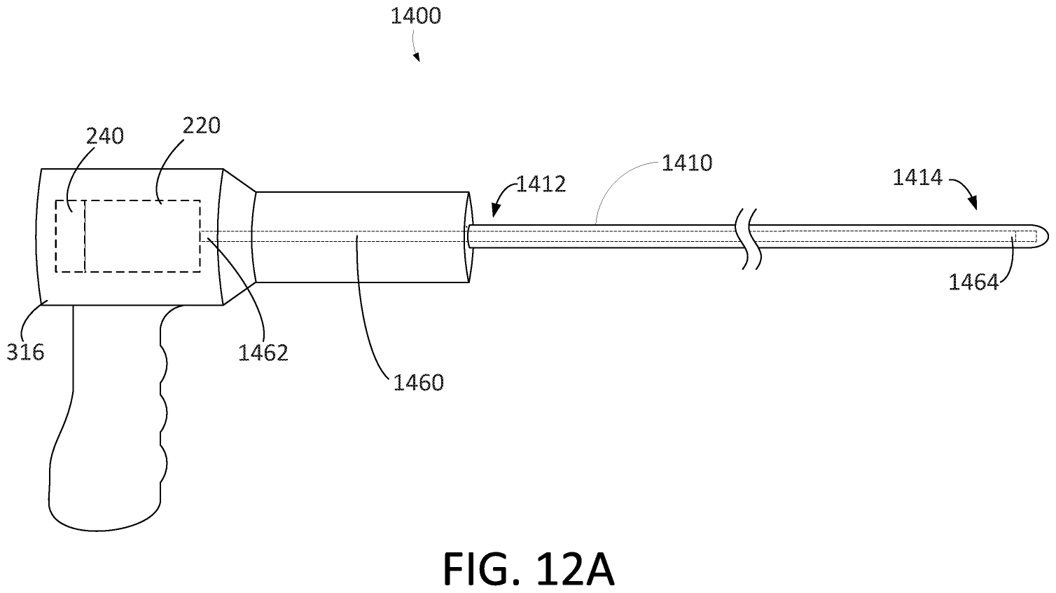

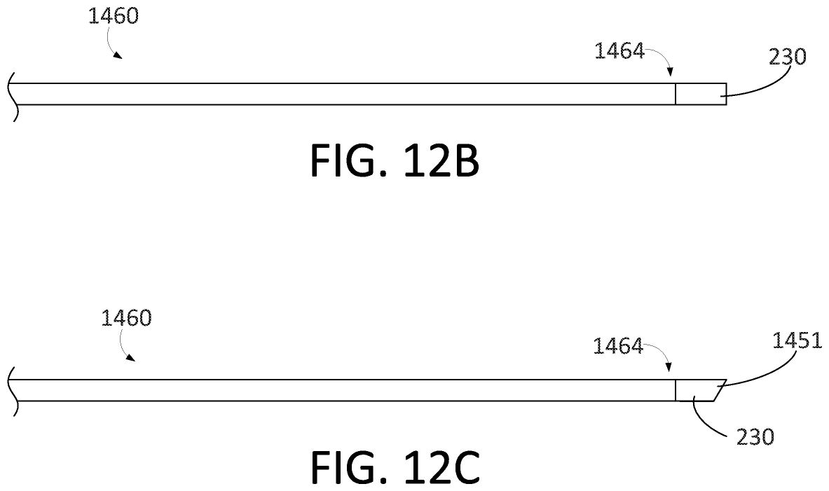

In some embodiments, the catheter 1400 can include an optical fiber 1460 as shown in FIGS. 12A-12C. Such a catheter 1400 can be used for photodynamic therapy. The catheter 1400 can be similar to those described elsewhere with respect to sonodynamic therapy except that it can include an optical fiber 1460 and an optical element 230 instead of a conductive pair 340 and an ultrasound transducer 303.

The optical fiber 1460 can be housed by the housing 1410 as shown in FIG. 12A. The optical fiber 1460 can extend between the proximal end 1412 and the distal end 1414 of the housing 1410. The optical fiber 1460 can have a first end and a second end 1464.

The first end 1462 of the optical fiber 1460 can be configured to be connected to a light supply 240. The light supply 240 can provide light to the optical element 230 via transmission by the optical fiber 1460 in some embodiments. In various instances, for example, the light supply 240 can provide continuous illumination to the optical element 230. The light supply 240 in some instances is dimmable, for example, to provide a range of spectrum of light to the optical element 230. In many embodiments, the light supply 240 can use either or both of AC and DC voltage sources.

In some embodiments, the catheter 1400 can include an optical element 230. The optical element 230 can be at the second end 1464 of the optical fiber 1460 as shown in FIG. 12B. The optical element 230 can be housed by the housing 1410. In many embodiments, the optical element 230 can be an electrical light (e.g., a laser diode, LED, halogen lamp, etc.). The optical element 230 may be integral to the optical fiber 1460 in some embodiments. In some embodiments, the optical element 230 can be separate and attachable to the optical fiber 1460.

The optical element 230 can be configured to emit light. The optical element 230 can emit light into the internal tissue of the patient. The optical element 230 can emit light during the minimally invasive procedure. Emission of light can facilitate treating the internal tissue of a patient. Light emitted by the optical element 230 can radiate beyond the housing 110.

In some embodiments, the optical element 230 can include a shaped tip 1461 of the second end 1464 of the optical fiber 1460 as shown in FIG. 12C. Though depicted as a particular size and shape, the shaped tip 1461 can be any suitable size and shape. In many embodiments, the shaped tip 1461 can be configured to modify the emission of light from the optical element 230. For example, in some embodiments, the profile of the emitted light can correspond to the geometry of the shaped tip 1461. In some embodiments, the shaped tip 1461 can be detachable from the optical fiber 1460. Some instances of the shaped tip 1461 can be interchangeable.

In many embodiments, the optical fiber 1460 may include a core surrounded by a cladding material. In some embodiments, the optical element 230 may include a structure that diffuses light. Such structure can be one or more grooves in the cladding of the optical fiber (e.g., a helical groove. In some embodiments, the optical element may be a tip of the second end 1464 of the optical fiber 1460. In some such embodiments, the tip can be beveled relative to the catheter axis. In some embodiments, the tip of the second end 1464 of the optical fiber 1460 can be oriented to emit light coaxially with a catheter axis. In some embodiments, the optical element 230 may include a mirror that faces the second end 1464 of the optical fiber 1460 and that is oriented at a non-zero angle with the catheter axis. In some instances, the mirror may be configured to reflect light emitted from the tip of the second end 1464 of the optical fiber 1460 into the internal tissue of the patient during a minimally invasive procedure. In some embodiments, the mirror may include a reflective surface (e.g., a flat reflective surface) that is configured to reflect light emitted from the tip of the second end 1464 of the optical fiber 1460. In some instances, the mirror may be coupled to the housing and pivotable about a pivot axis (e.g., perpendicular to the catheter axis) relative to the housing.

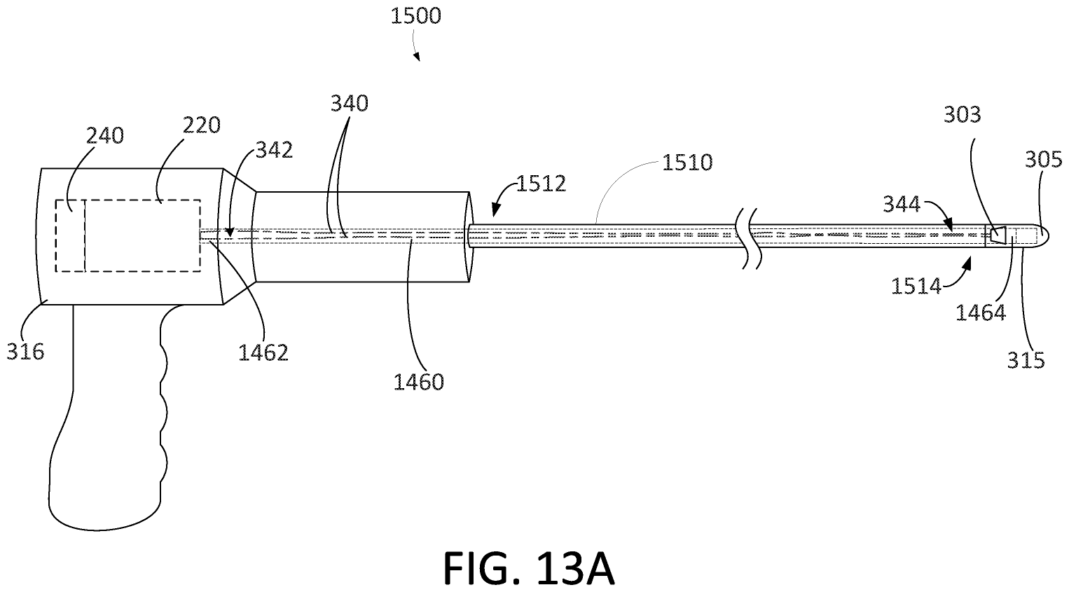

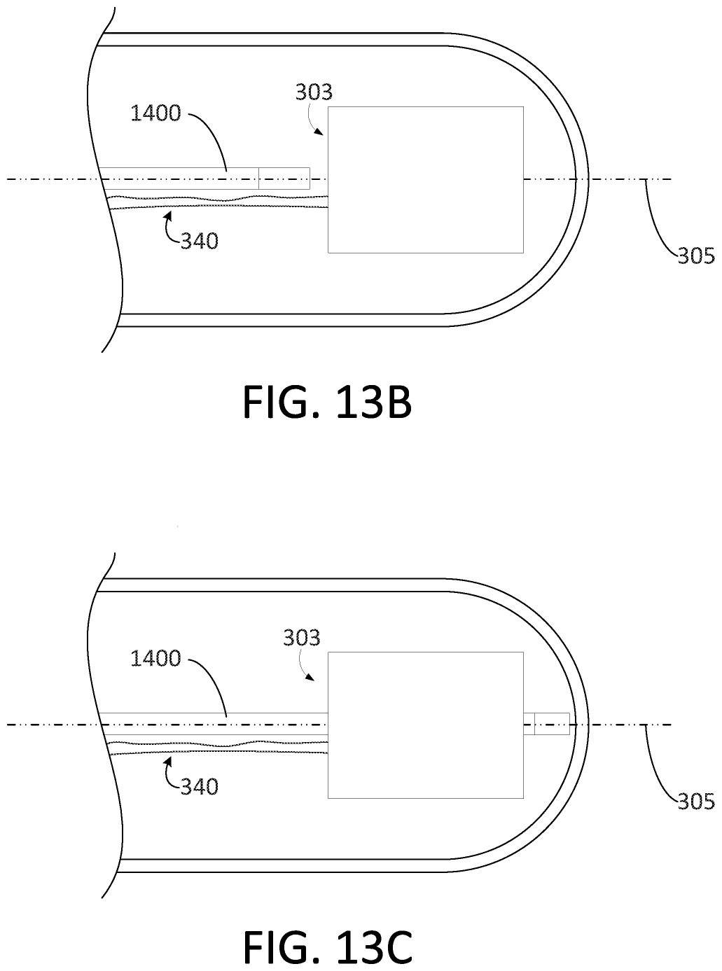

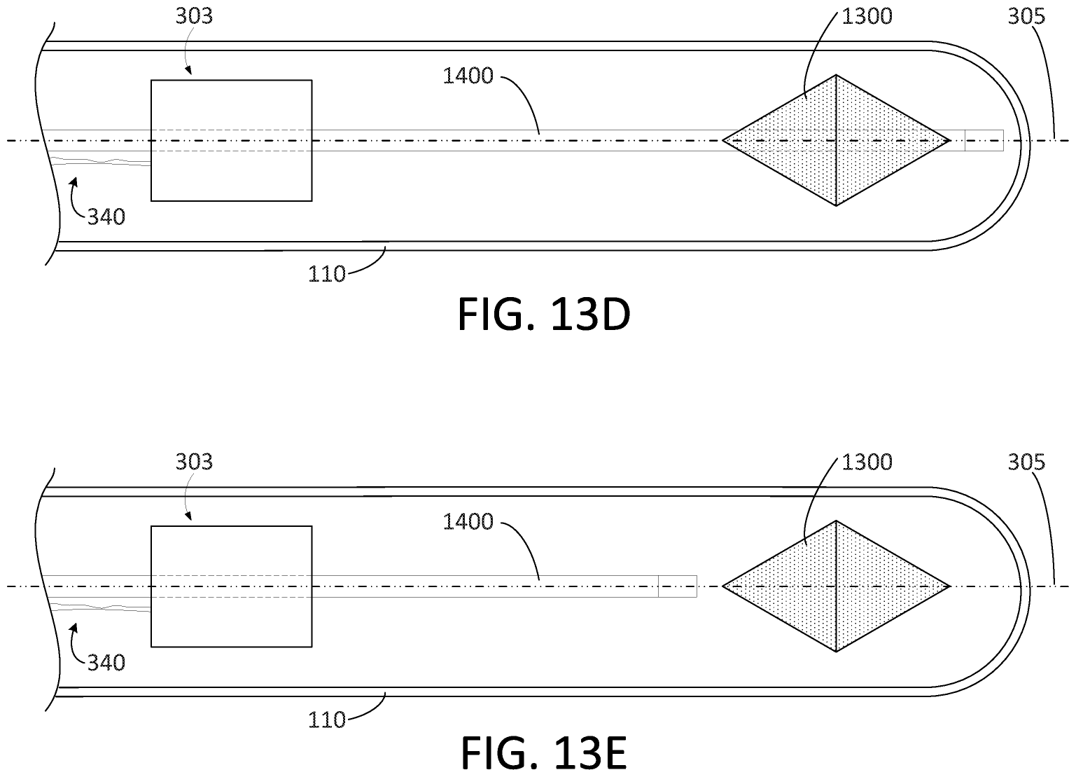

In many embodiments, the catheter 1500 can be configured to emit ultrasound energy and light as shown in FIGS. 13A-13E. Such a catheter 1500 can be used in performing photo-sonodynamic therapy. These catheters 1500 can be similar to those described elsewhere herein except that the catheter 1500 can include an ultrasound transducer 303, an optical fiber 1460, and an optical element 230. These components of the catheter 1500 can be similar to those described elsewhere herein. Such a catheter 1500 may include any combination or none of the accompanying features (e.g., transducer housing 315, acoustic element 1300, shaped tip 1461, etc.) described elsewhere herein. In some embodiments, the ultrasound transducer 303 can be proximal to both the optical element 230 and an acoustic element 1300 if provided, distal to both the optical element 230 and the acoustic element 1300 if provided, or proximal to one and distal to the other.

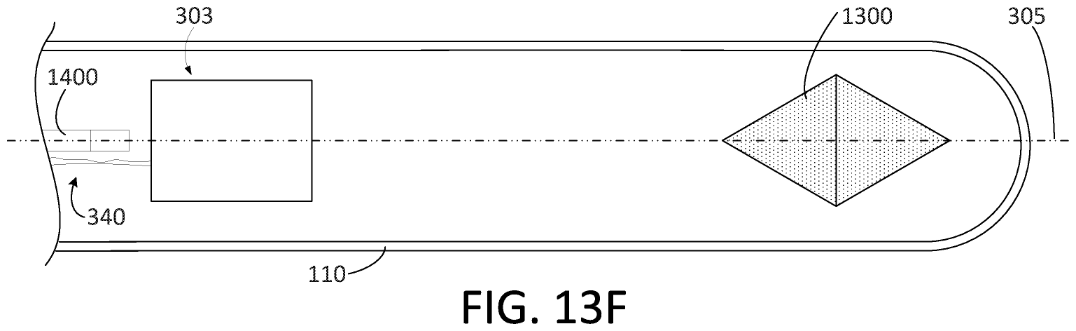

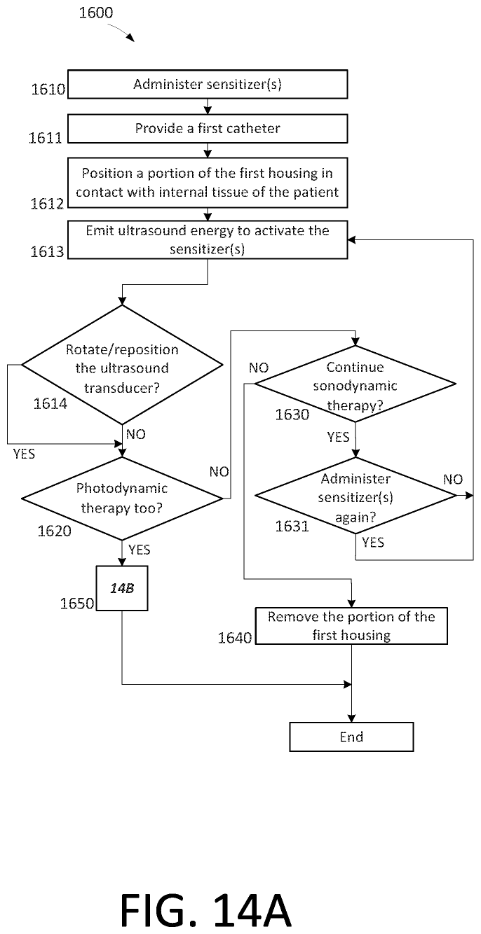

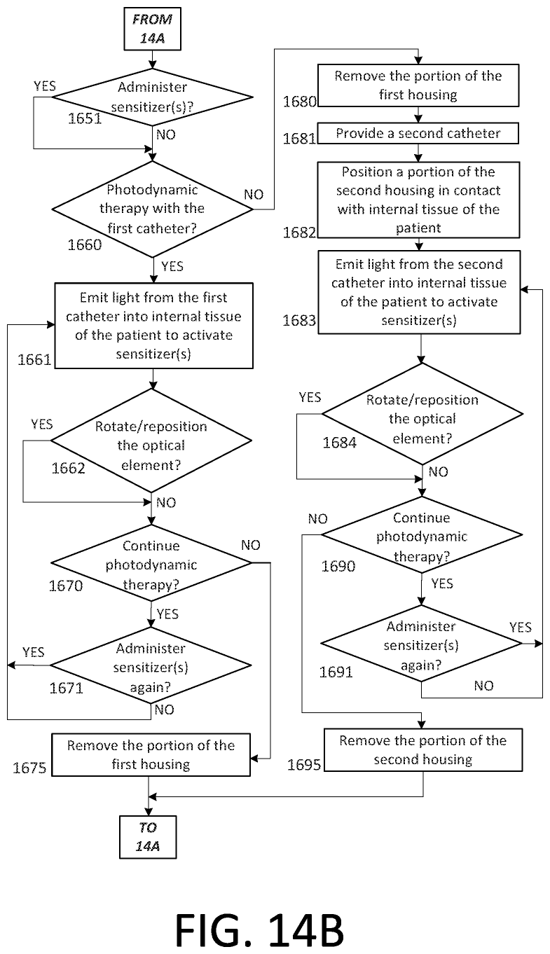

Ultrasound energy, light, or any combination thereof can be emitted into the internal tissue of a patient using a method 1600 as shown in FIGS. 14A-14B. Various processes have been described in which one or more catheters can be used in performing medical procedures using any combination of ultrasound energy or light. In some embodiments, these steps can be aggregated into a multi-step process in order to treat the internal tissue of a patient (e.g., brain tissue). One skilled in the art can appreciate that at least some of the steps may be omitted, rearranged, or modified without departing from the scope of this disclosure.

As shown in FIG. 14A, in various embodiments, a patient can be administered one or more sensitizers 1610. The method 1600 can include administering one or more sensitizers to a patient. In many embodiments, the method 1600 can include administering a second sensitizer to the patient. The sensitizers can increase the sensitivity of exposure to sound or light (e.g., sonosensitizers and photosensitizes) such that when they are activated, they can kill tissue that has the increased sensitivity to sound or light. The sensitizers can be configured to increase the sensitivity of undesirable (e.g., cancerous, malignant, etc.) tissue. For example, the sensitizer can saturate undesirable tissue and not saturate desirable tissue such that the tissue that is saturated with sensitizer can be highly reactive to exposure to sound and light at a particular frequency or spectrum.

Many embodiments of the method 1600 can include providing a first catheter 1611. The first catheter can be similar to those described elsewhere herein. For example, the first catheter can be configured to perform any of sonodynamic, photodynamic, or photo-sonodynamic therapy.

The method 1600 can include manipulating the position of the first catheter. In such embodiments, a user can position a portion of the first housing to be in contact with the internal tissue of the patient 1612. In many embodiments, positioning the portion of the first housing in contact with internal tissue of the patient 1612 can include positioning the portion of the first housing intracranially in contact with brain tissue of the patient. In some embodiments, positioning the portion of the first housing intracranially in contact with brain tissue of the patient can include inserting the portion of the first housing through a burr hole. Inserting the portion of the first housing through a burr hole can put the housing into contact with the brain tissue. In some embodiments, positioning the portion of the first housing in contact with internal tissue of the patient 1612 can include using a stereotactic guidance system in connection with a location marker. For example, positioning the portion of the first housing may include using a stereotactic guidance system in connection with markings used for measuring a distance from a reference point. In some embodiments, positioning the portion of the first housing in contact with internal tissue of the patient 1612 can include using a stereotactic guidance system in connection with an adjustable depth stop that is configured to slide over the first housing and to be locked in different locations.

The first catheter in several embodiments can emit ultrasound energy to activate the one or more sensitizers 1613. The method 1600 can include emitting ultrasound energy from the first emitting surface of the ultrasound transducer 1613 as similarly described elsewhere herein. In some instances, the ultrasound transducer can emit the ultrasound energy in a continuous waveform. In some embodiments, the ultrasound transducer can pulse the ultrasound energy (e.g., emit the ultrasound energy in square pulses). The emitted ultrasound energy can activate the sensitizer(s). In some embodiments, emitting ultrasound energy from the first emitting surface of the ultrasound transducer as described elsewhere herein can activate one or more sensitizers that have been administered to the patient. In some embodiments, multiple sensitizers are activated by emitting ultrasound energy at the same frequency while other embodiments can have the multiple sensitizers activated at different frequencies.

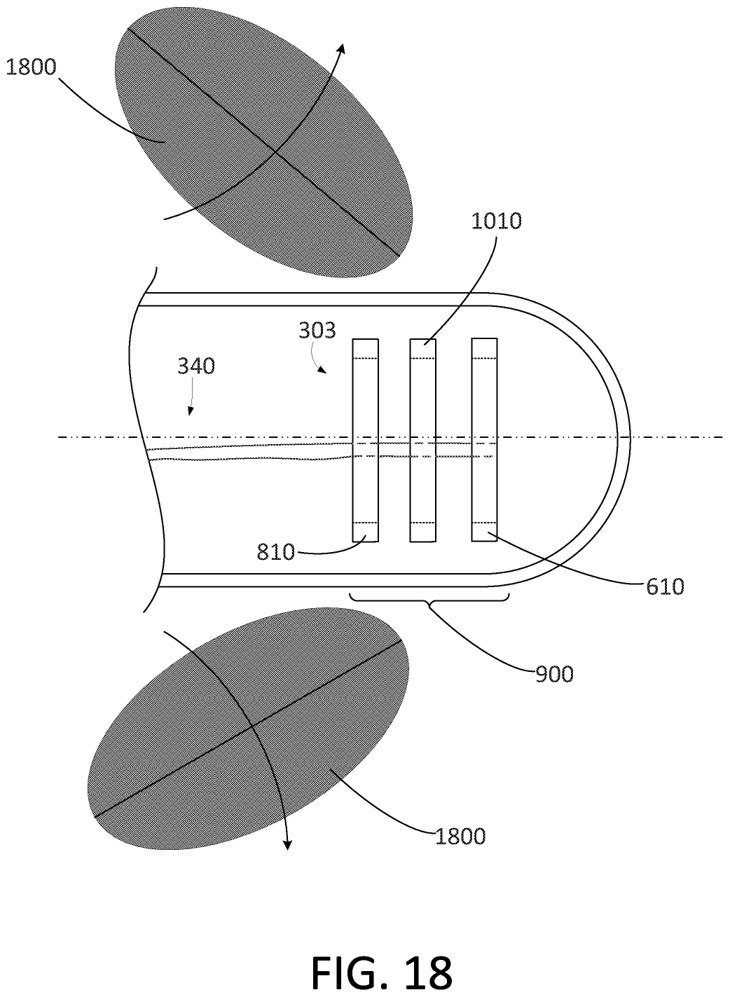

FIG. 18 shows an embodiment in which the ultrasound transducer 303 includes multiple ultrasound transducers 303, an embodiment similarly described elsewhere herein. In such embodiments, step 1613 of the method 1600 of FIG. 14A can include emitting ultrasound energy from the multiple ultrasound transducers 303 into the internal tissue of the patient. Referring again to FIG. 14A, emitting ultrasound to activate the one or more sensitizers can be done using a variety of the catheters disclosed herein. For example, emitting ultrasound energy from the multiple ultrasound transducers into the internal tissue of the patient can activate the sensitizer(s) using a beamforming technique 1800. Beamforming, for example, can create a series constructive and destructive interface to focus the emitted ultrasound energy in a particular direction as emitted ultrasound energy extends beyond the housing 110.

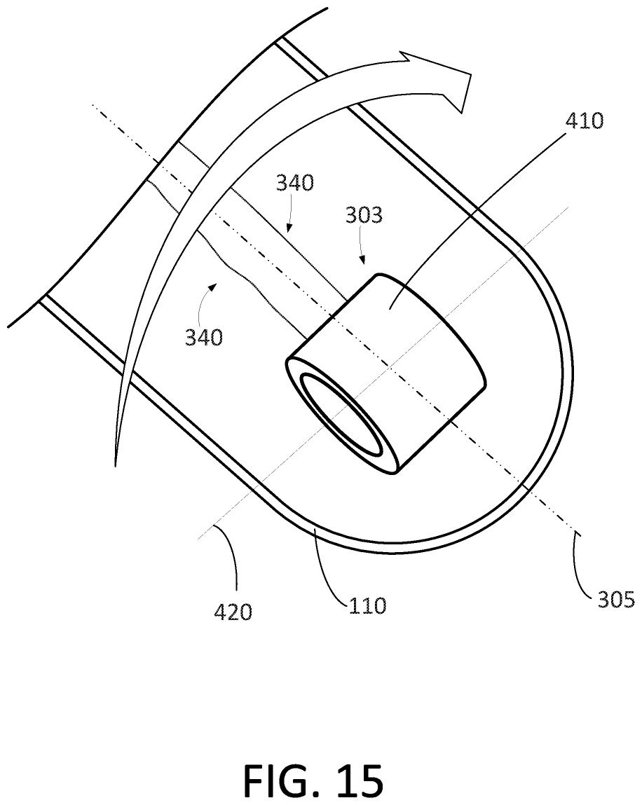

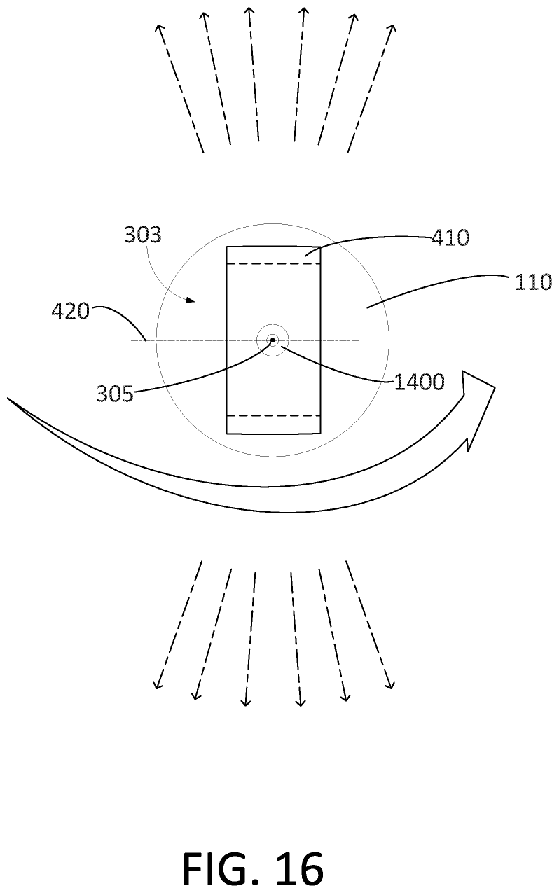

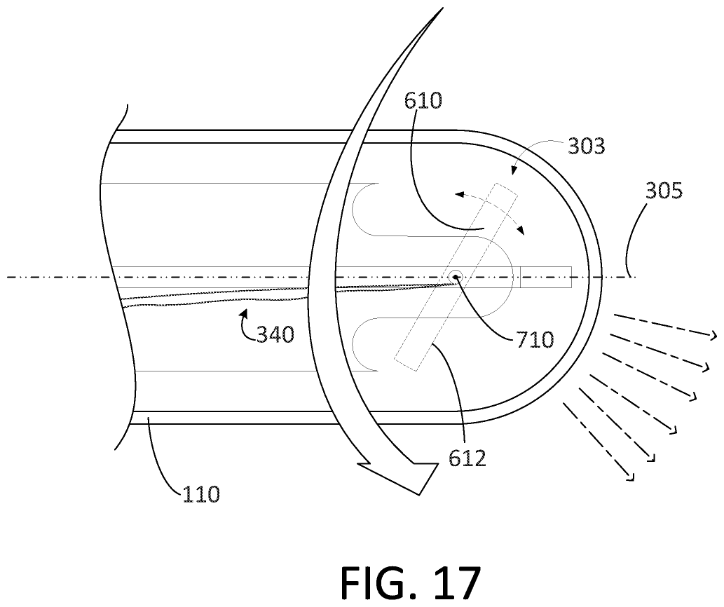

Referring again to FIG. 14A, in many embodiments, the method 1600 can include rotating and/or repositioning the ultrasound transducer 1614. As can be seen, for example, in FIGS. 15, 16, and 17, in some embodiments, the ultrasound transducer 303 can be rotated about the catheter axis 305. The ultrasound transducer 303 in many instances can rotate about the catheter axis 305 relative to the portion of the housing 110. In some embodiments, the ultrasound transducer 303 can be rotated while the portion of the housing 110 is in contact with the internal tissue of the patient. In many embodiments, the ultrasound transducer 303 and the portion of the housing 110 can rotate together. In some embodiments, the ultrasound transducer 303 and the portion of the housing 110 can rotate together about the catheter axis 305. In some embodiments, the ultrasound transducer may be repositioned via translation.

Referring again to FIG. 14A, the user can make several determinative decisions on how to proceed with a minimally invasive procedure. The user may decide to rotate the ultrasound transducer and/or to reposition the ultrasound transducer via translation 1614. The user can decide whether to perform photodynamic therapy in addition to sonodynamic therapy 1620. The user can decide whether to continue sonodynamic therapy 1630.

If a user determines that photodynamic therapy is not needed and that sonodynamic therapy is complete, the user may bring the minimally invasive procedure to completion. The method 1600 can include removing the first catheter. In some embodiments, the method 1600 can include removing the portion of the first housing from contact with internal tissue of the patient 1640. In some embodiments, the method 1600 includes removing the portion of the first housing from the brain tissue of a patient.

As noted, the user can decide to continue sonodynamic therapy 1630 in various instances. In some embodiments, administering a sensitizer to the patient can be done multiple times 1631 as shown in FIG. 14A. In many embodiments, administering a sensitizer to the patient can include administering the sensitizer to the patient multiple times. This can continue sonodynamic therapy in some embodiments of the method 1600. In such embodiments, the sonodynamic therapy can continue by emitting ultrasound from the first catheter 1613 as described elsewhere herein. The user in some embodiments can choose whether to rotate/reposition the ultrasound transducer or not 1614 as described elsewhere herein, for example, during emission of ultrasound energy. This process can continue, for instance, until the user is satisfied with the sonodynamic therapy or needs to stop the process beforehand. In such instances, removing the first housing 1640 as described elsewhere herein can bring the minimally invasive procedure to completion.

Various embodiments of the method 1600 can have the user include performing photo-sonodynamic therapy 1620. In several embodiments, photodynamic therapy can be performed by a catheter 1650 as shown in FIG. 14B. In many instances, photodynamic therapy can occur after sonodynamic therapy is ended by the user. In some instances, sonodynamic therapy can be performed multiple times before photodynamic therapy. In some instances, photodynamic therapy may occur before sonodynamic therapy. In some instances, photodynamic therapy can be performed multiple times before sonodynamic therapy. Referring to FIG. 14B, in any of these instances, the user can choose to administer one or more sensitizers to the patient 1651 before proceeding with photodynamic therapy.

In many embodiments, the user can decide whether to perform photodynamic therapy or not. The user can decide to perform photodynamic therapy in many embodiments with the first catheter 1660. The method can include emitting light into the internal tissue of the patient (e.g., brain tissue) from the first catheter 1661 as similarly described elsewhere herein. Emitting light into the internal tissue of the patient can activate the sensitizer(s). In some instances, emitting light can activate one or more of multiple sensitizers. In some embodiments, multiple sensitizers are activated by emitting light at the same wavelength while other embodiments can have the multiple sensitizers activated at different wavelengths.

An illustrative method can include providing one or more catheters. In many embodiments, photodynamic therapy can be performed by the same catheter as is used to perform sonodynamic therapy. In such instances, light can be emitted from the first catheter to activate the one or more sensitizers 1661. The user can rotate and/or reposition the optical element 1662 similar to the manner as described elsewhere herein for the ultrasound transducer. In some instances, the optical element can be rotated and/or repositioned while the first catheter is emitting light. In some embodiments, the user can choose whether to rotate and/or reposition the optical element 1662 or not.

The user can decide whether to continue photodynamic therapy 1670 or not in various instances. In some embodiments, continuing photodynamic therapy can include administering a sensitizer to the patient multiple times 1671 as shown in FIG. 14B. In many embodiments, one or more sensitizers may be administered to the patient multiple times. In such embodiments, the photodynamic therapy can continue by emitting light from the first catheter 1661 as described elsewhere herein. The user in some embodiments can choose whether to rotate and/or reposition the optical element 1662 or not as described elsewhere herein, for example, during the emission of light. The user can decide to continue photodynamic therapy 1670 of not, for instance, until the user is satisfied with the photodynamic therapy or needs to stop the process beforehand. In such instances, removing the first housing 1675 as described elsewhere herein can bring the minimally invasive procedure to completion.

In several embodiments, photodynamic therapy can be performed with the second catheter. For example, the user can decide to use a second catheter to perform photodynamic therapy instead of the first catheter 1660. The method can include providing a second catheter 1681. The second catheter can be similar to those described elsewhere herein. The second catheter in an illustrative method can have a different configuration than the first catheter. For example, in some instances, the second catheter can include a second housing, an optical fiber, and an optical element as disclosed elsewhere herein while the first catheter can include a first housing, a conductive pair, and an ultrasound transducer as disclosed elsewhere herein. In all of these instances, removing the first housing can allow the user to position the second catheter to perform photodynamic therapy.

The method can include manipulating the position of the second catheter. In some instances, the method can include positioning a portion of the second housing in contact with internal tissue of the patient 1682. In some embodiments, removing the portion of the first housing from contact with internal tissue of the patient 1680 can be before positioning the portion of the second housing in contact with internal tissue of the patient 1682. In some embodiments, positioning the portion of the second housing in contact with internal tissue of the patient 1682 can be before removing the portion of the first housing from contact with internal tissue of the patient. In some instances, performing photodynamic therapy can occur before performing sonodynamic therapy. In some instances, performing sonodynamic therapy can occur before performing photodynamic therapy.

The method can include emitting light into the internal tissue of the patient from the second catheter 1683 as similarly described elsewhere herein. Emitting light into the internal tissue of the patient can activate the sensitizer(s). This can continue photodynamic therapy in some embodiments of the method. In such embodiments, the photodynamic therapy can continue by emitting light from the first catheter as described elsewhere herein. The user in some embodiments can choose whether to rotate and/or reposition the ultrasound transducer 1684 or not as described elsewhere herein, for example, during emission of emission of light.

If it is determined that photodynamic therapy need not continue, the method 1600 can include removing the second catheter. In some embodiments, the method 1600 can include removing the portion of the second housing from contact with internal tissue of the patient 1695. In embodiments in which photodynamic therapy is performed before sonodynamic therapy, the photodynamic therapy catheter can be removed before positioning the sonodynamic therapy catheter. In some embodiments, positioning the portion of the second housing in contact with internal tissue of the patient 1682 can be before removing the portion of the first housing from contact with internal tissue of the patient 1680.

In certain embodiments, the user can continue photodynamic therapy 1690, for instance, until the user is satisfied with the photodynamic therapy or needs to stop the process beforehand. In such instances, the user can decide whether or not to administer one or more sensitizers to the patient again 1691. In some embodiments, administering a sensitizer to the patient can be done multiple times as described elsewhere herein. This can continue photodynamic therapy in some embodiments of the method. In such embodiments, the photodynamic therapy can continue by emitting light from the second catheter 1683 as described elsewhere herein. The user in some embodiments can choose whether to rotate and/or reposition the optical element 1684 or not as described elsewhere herein, for example, during the emission of light. When the user determines that photodynamic therapy is complete, the user may remove the second housing 1695, which may bring the minimally invasive procedure to completion.

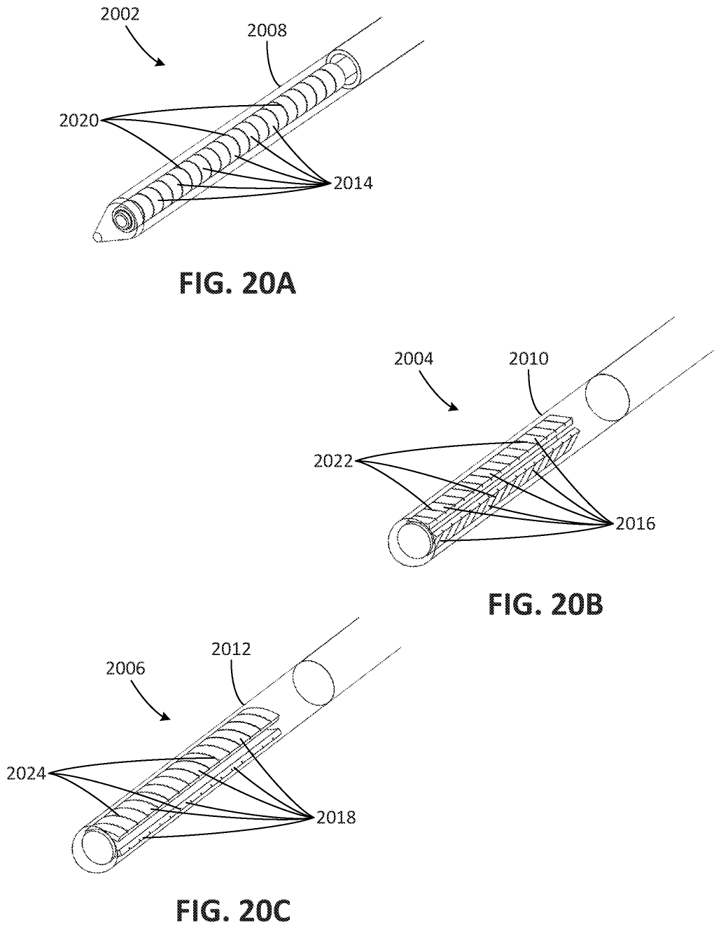

FIGS. 20A-20C show examples of catheters 2002, 2004, 2006 that can be used for purposes of minimally invasive treatment according to techniques and method discussed herein. Each of the catheters 2002, 2004, 2006 can include a housing 2008, 2010, 2012 like those discussed herein. FIGS. 20A-20C show the distal ends of the housings 2008, 2010, 2012, at least a portion of which may be configured to be positioned in contact with internal tissue of a patient during a minimally invasive procedure that involves a sensitizer. Each catheter 2002, 2004, 2006 may include ultrasound transducers 2014, 2016, 2018 housed by the respective housing 2008, 2010, 2012. The catheters 2002, 2004, 2006 may include conductive pairs housed by the housing and connected to each of the ultrasound transducers 2014, 2016, 2018 (like the configuration of FIG. 10D). In this manner, each of the ultrasound transducers 2014 of catheter 2002 may be configured to emit ultrasound energy independently of one another, each of the ultrasound transducers 2016 of catheter 2004 may be configured to emit ultrasound energy independently of one another, and each of the ultrasound transducers 2018 of catheter 2006 may be configured to emit ultrasound energy independently of one another.

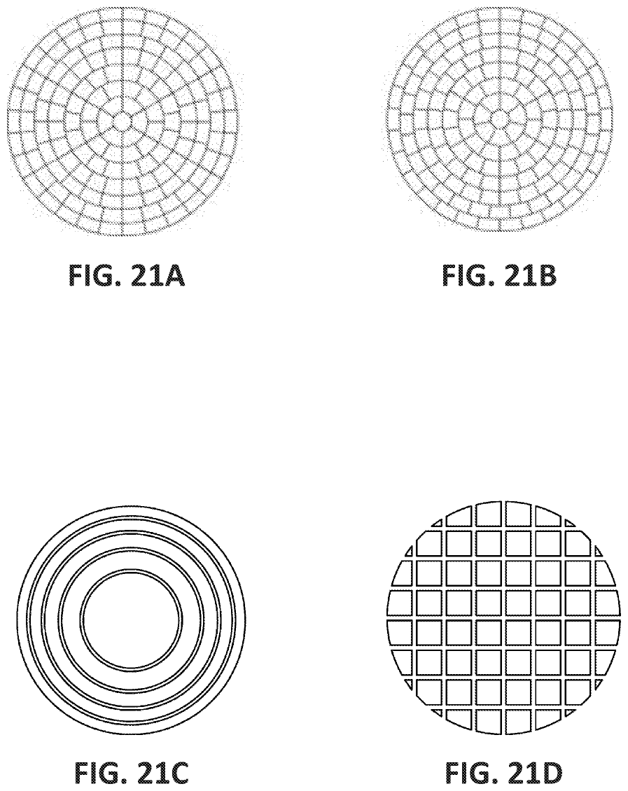

Ultrasound transducers used in catheters for minimally invasive treatments can have various structural configurations. In some embodiments, each ultrasound transducer can be physically and electrically separate from one another (e.g., FIG. 10D). In some embodiments, such as those shown in FIGS. 20A-20C, each ultrasound transducer may be electrically separate from one another but physically part of the same structure. The ultrasound transducers 2014 of FIG. 20A are physically part of the same tube but electrically distinct from one another. Relief cuts 2020 can serve to mechanically isolate the ultrasound transducers 2014 from one another. In some instances, tubular structures may be especially amenable to efficient manufacturing techniques. The ultrasound transducers 2016 of FIG. 20B are physically part of one of three physically separate flat arrays, such that each array has multiple ultrasound transducers 2016, with each ultrasound transducer 2016 being electrically independent from each other. Three arrays are shown, but any suitable number of arrays may be used. Relief cuts 2022 can serve to mechanically isolate the ultrasound transducers 2016 from one another. The ultrasound transducers 2018 of FIG. 20C are physically part of one of two physically separate curved arrays, such that each array has multiple ultrasound transducers 2018, with each ultrasound transducer 2018 being electrically independent from each other. Relief cuts 2024 can serve to mechanically isolate the ultrasound transducers 2018 from one another. Two curved arrays are shown, but any suitable number of curved arrays may be used. The three configurations shown in FIGS. 20A-20C are illustrative. In many embodiments, the ultrasound transducers of an array may be electrically stimulated together, with each array emitting ultrasound energy independently of each other array. In some embodiments, ultrasound transducers may take the form of a two-dimensional array (see FIGS. 21A-21D). FIGS. 21A-21C show annular arrays. FIG. 21D shows a rectilinear array. In some embodiments (e.g., FIGS. 21A-21B), each ultrasound transducer in an annular array may have the same area. In some embodiments, multiple physically separate ultrasound transducers may be joined together (e.g., via glue) to form a single array that has multiple electrically independent ultrasound transducers.

Catheters 2002, 2004, 2006 like those of FIGS. 20A-20C can operate in a manner similar to other catheters described herein. For example, the ultrasound transducers 2014, 2016, 2018 may emit ultrasound energy (e.g., independently from one another) into internal tissue (e.g., brain tissue) of a patient to activate one or more sensitizers. The ultrasound energy emitted by the ultrasound transducers 2014, 2016, 2018 may reach a target tissue depth at a relatively low intensity as discussed herein. In some embodiments, catheters with multiple electrically independent ultrasound transducers, like those of FIGS. 20A-20C, may be able to adjust the pattern of emitted ultrasound energy. For example, rather than emitting ultrasound in a pattern having a main lobe and two side lobes, multiple ultrasound transducers may be electrically stimulated to different degrees in order to reduce or eliminate the side lobes and bolster the main lobe. In another example, neighboring ultrasound transducers may be electrically stimulated to an ascending or descending degree such that the catheter's overall ultrasound energy pattern is at a specified angle relative to the catheter axis. In another example, ultrasound transducers can be controlled to emit ultrasound energy such that the strength of the field along the main lobe decays slowly. This beamforming functionality provides significant advantages in being able to supply ultrasound energy to internal tissue that may otherwise be difficult to access.

In catheter embodiments with at least three ultrasound transducers that are each connected to a power supply by its own conductive pair, ultrasound energy may be emitted from first, second, and third ultrasound transducers into internal tissue of a patient to activate one or more sensitizers, with the ultrasound energy reaching a target tissue depth at a temporal average intensity of less than 50 W/cm². In some such embodiments, the first and third (outer) ultrasound transducers may be electrically stimulated at a different amplitude and/or phase than the second (middle) ultrasound transducer to create an ultrasound energy pattern with reduced side lobes and a bolstered main lobe. In some embodiments, the first ultrasound transducer may be electrically stimulated later than the second (middle) transducer and even later than the third (opposite end) transducer to create an angled ultrasound energy pattern. In some embodiments, an ultrasound energy field strength along a main lobe may be designed to decay slowly along its length. The ultrasound energy field strength along each point in a path of a main lobe may vary by no more than 20 dB until reaching the target tissue depth.

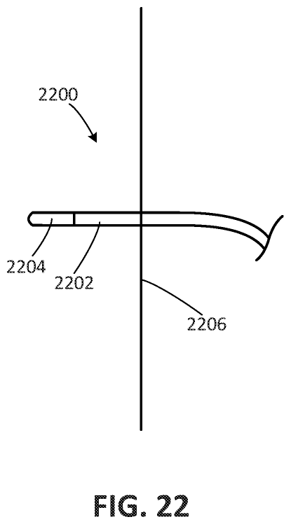

FIG. 22 shows a catheter 2200 that has similar characteristics and functions similarly to catheters discussed herein. The housing of the catheter 2200 may include a sheath 2202 that defines a lumen that extends along a catheter axis. One or more conductive pairs may extend within the lumen as described elsewhere herein. The housing of the catheter 2200 may include a transducer housing 2204. The transducer housing 2204 may house one or more ultrasound transducers. In some embodiments, the sheath 2202 may be made of a different material than the transducer housing 2204. In some such embodiments, the material of which the sheath 2202 is made may be more flexible than the material of which the transducer housing 2204 is made. The sheath 2202 may be sized to extend from within a patient 2206 to outside the patient 2206 during a minimally invasive procedure, such as those discussed elsewhere herein.

Various examples have been described with reference to certain disclosed embodiments. The embodiments are presented for purposes of illustration and not limitation. One skilled in the art will appreciate that various changes, adaptations, and modifications can be made without departing from the scope of the invention.

What is claimed is:

1. A catheter for minimally invasive internal treatment, comprising:
a housing including a proximal end and a distal end, a portion of the housing being configured to be positioned in contact with internal tissue of a patient during a minimally invasive procedure that involves a sensitizer;
first and second conductive pairs housed by the housing and extending between the proximal end and the distal end, each of the first and second conductive pairs having a first end configured to be connected to a power supply and a second end; and
first and second ultrasound transducers housed by the housing, the first ultrasound transducer being connected to the second end of the first conductive pair, the second ultrasound transducer being connected to the second end of the second conductive pair, the first and second ultrasound transducers being configured to emit ultrasound energy into the internal tissue of the patient such that the ultrasound energy reaches a target tissue depth at a temporal average intensity of less than 50 W/cm² and such that the ultrasound energy activates the sensitizer during the minimally invasive procedure.

2. The catheter of claim 1, wherein the portion of the housing is configured to be positioned intracranially in contact with brain tissue of the patient during the minimally invasive procedure, and wherein the first and second ultrasound transducers are configured to emit ultrasound energy into the brain tissue of the patient such that the ultrasound energy reaches a target brain tissue depth at a temporal average intensity of less than 50 W/cm² and such that the ultrasound energy activates the sensitizer during the minimally invasive procedure.

3. The catheter of claim 1, further comprising an acoustic element housed by the housing, the acoustic element being configured to modify a direction at which ultrasound energy emitted by the first and second ultrasound transducers enters the internal tissue of the patient during the minimally invasive procedure.

4. The catheter of claim 3, wherein the acoustic element comprises an acoustic lens in contact with the first ultrasound transducer and/or the second ultrasound transducer to modify a focus of an acoustic wavefront formed by the emitted ultrasound energy.

5. The catheter of claim 3, wherein the acoustic element comprises an acoustic prism.

6. The catheter of claim 1, wherein the first and second ultrasound transducers are configured to emit ultrasound energy into the internal tissue of the patient such that the ultrasound energy reaches a target tissue depth at a temporal average intensity of less than 5 W/cm² and such that the ultrasound energy activates the sensitizer during the minimally invasive procedure.

7. The catheter of claim 1, wherein the housing comprises a sheath defining a lumen that extends along a catheter axis, the first and second conductive pairs extending within the lumen, the housing further comprising a transducer housing that houses the first and second ultrasound transducers.

8. The catheter of claim 7, wherein the sheath is made of a first material and the transducer housing is made of a second material, the first material being more flexible than the second material.

9. The catheter of claim 8, wherein the sheath is sized to extend from within the patient to outside the patient during the minimally invasive procedure.

10. The catheter of claim 1, wherein the portion of the housing has a cross-sectional area of less than 154 mm².

11. The catheter of claim 1, wherein the housing is made of a material having an acoustic impedance similar to that of the internal tissue of the patient.

12. The catheter of claim 1, further comprising an acoustic transmission material positioned between the first ultrasound transducer and/or the second ultrasound transducer and where ultrasound energy exits the housing into the internal tissue of the patient.

13. The catheter of claim 12, wherein the housing comprises a transducer housing that houses the first and second ultrasound transducers, and wherein the acoustic transmission material comprises a fluid couplant that fills the transducer housing.

14. The catheter of claim 12, wherein the acoustic transmission material comprises one or more acoustic matching layers coated on the first and second ultrasound transducers.

15. The catheter of claim 12, wherein the acoustic transmission material comprises an elastic boot.

16. The catheter of claim 1, wherein the housing further includes a location marker for use with a stereotactic guidance system.

17. The catheter of claim 1, further comprising an adjustable depth stop configured to slide over the housing and to be locked in different locations for use with a stereotactic guidance system.

18. The catheter of claim 1, wherein the housing includes markings measuring a distance from a reference point for use with a stereotactic guidance system.

19. A method comprising:
administering a first sensitizer to a patient;
providing a first catheter that includes:
a first housing comprising a proximal end and a distal end and defining a first catheter axis,
first and second conductive pairs housed by the first housing and extending between the proximal end and the distal end, each of the first and second conductive pairs having a first end connected to a power supply and a second end, and
first and second ultrasound transducers housed by the first housing, the first ultrasound transducer being connected to the second end of the first conductive pair, the second ultrasound transducer being connected to the second end of the second conductive pair;
positioning a portion of the first housing in contact with internal tissue of the patient; and
emitting ultrasound energy from the first and second ultrasound transducers into the internal tissue of the patient to activate the first sensitizer, the ultrasound energy reaching a target tissue depth at a temporal average intensity of less than 50 W/cm².

20. The method of claim 19, wherein emitting ultrasound energy from the first and second ultrasound transducers comprises emitting the ultrasound energy in a continuous waveform.

21. The method of claim 19, wherein emitting ultrasound energy from the first and second ultrasound transducers comprises emitting sine wave bursts of the ultrasound energy.

22. The method of claim 21, wherein emitting ultrasound energy from the first and second ultrasound transducers comprises emitting pulses of the ultrasound energy.

23. The method of claim 19, wherein administering the first sensitizer to the patient comprises administering the first sensitizer to the patient multiple times.

24. The method of claim 19, further comprising administering a second sensitizer to the patient, wherein emitting ultrasound energy from the first and second ultrasound transducers into the internal tissue of the patient activates both the first sensitizer and the second sensitizer.

25. The method of claim 19, wherein positioning the portion of the first housing in contact with internal tissue of the patient comprises positioning the portion of the first housing intracranially in contact with brain tissue of the patient, and wherein emitting ultrasound energy from the first and second ultrasound transducers into the internal tissue of the patient comprises emitting ultrasound energy into the brain tissue of the patient.

26. The method of claim 25, wherein positioning the portion of the first housing intracranially in contact with brain tissue of the patient comprises inserting the portion of the first housing through a burr hole into contact with the brain tissue.

27. The method of claim 19, wherein emitting ultrasound energy from the first and second ultrasound transducers into the internal tissue of the patient comprises using a beamforming technique.

28. The method of claim 19, wherein the first catheter further includes an acoustic element housed by the first housing, and wherein emitting ultrasound energy from the first and second ultrasound transducers into the internal tissue of the patient comprises modifying a direction at which ultrasound energy emitted by the first and second ultrasound transducers enters the internal tissue of the patient with the acoustic element.

29. The method of claim 28, wherein the acoustic element comprises an acoustic lens in contact with the first ultrasound transducer and/or the second ultrasound transducer, and wherein modifying the direction at which ultrasound energy emitted by the first and second ultrasound transducers enters the internal tissue of the patient comprises modifying a focus of an acoustic wavefront formed by the emitted ultrasound energy with the acoustic lens.

30. The method of claim 28, wherein the acoustic element comprises an acoustic prism.

31. The method of claim 19, wherein the ultrasound energy reaches the target tissue depth at a temporal average intensity of less than 5 W/cm².

32. The method of claim 19, wherein the first housing of the first catheter comprises a sheath defining a lumen that extends along a first catheter axis, the first and second conductive pairs extending within the lumen, the first housing of the first catheter further comprising a transducer housing that houses the first and second ultrasound transducers.

33. The method of claim 32, wherein the sheath is made of a first material and the transducer housing is made of a second material, the first material being more flexible than the second material.

34. The method of claim 33, further comprising temporarily securing the sheath to the patient while the transducer housing is in contact with the internal tissue of the patient.

35. The method of claim 19, wherein the first housing further includes a location marker, and wherein positioning the portion of the first housing comprises using a stereotactic guidance system in connection with the location marker.

36. The method of claim 19, further comprising providing an adjustable depth stop that is configured to slide over the housing and to be locked in different locations, wherein positioning the portion of the first housing comprises using a stereotactic guidance system in connection with the adjustable depth stop.

37. The method of claim 19, wherein the housing further includes markings measuring a distance from a reference point, and wherein positioning the portion of the first housing comprises using a stereotactic guidance system in connection with the markings.

38. The method of claim 19,
wherein the first catheter further comprises:
a third conductive pair housed by the first housing and extending between the proximal end and the distal end, the third conductive pair also having a first end connected to the power supply and a second end, and
a third ultrasound transducer housed by the first housing, the third ultrasound transducer being connected to the second end of the third conductive pair, the second ultrasound transducer being positioned between the first and third ultrasound transducers, and
wherein the method further comprises emitting ultrasound energy from the first, second, and third ultrasound transducers into the internal tissue of the patient to activate the first sensitizer, the ultrasound energy reaching the target tissue depth at the temporal average intensity of less than 50 W/cm².

39. The method of claim 38, wherein emitting ultrasound energy from the first, second, and third ultrasound transducers into the internal tissue of the patient comprises electrically stimulating the second ultrasound transducer with a different amplitude and/or phase relative to the first ultrasound transducer or the third ultrasound transducer to create an ultrasound energy pattern with reduced side lobes and a bolstered main lobe.

40. The method of claim 38, wherein emitting ultrasound energy from the first, second, and third ultrasound transducers into the internal tissue of the patient comprises electrically stimulating the first ultrasound transducer later than the second ultrasound transducer and even later than the third ultrasound transducer to create an angled ultrasound energy pattern.

41. The method of claim 38, wherein emitting ultrasound energy from the first, second, and third ultrasound transducers into the internal tissue of the patient comprises causing an ultrasound energy field strength along each point in a path of a main lobe to vary by no more than 20 dB until reaching the target tissue depth.
